(19) United States
(12) Patent Application Publication
(10) Pub. No.: US 2007/0173545 A1
(43) Pub. Date: Jul. 26, 2007

(54) Activation of hypoxia-inducible gene expression

(76) Inventors: Ajay Verma, Harleysville, PA (US); Huasheng Lu, Boyds, MD (US); Robert Forbes, Princeton, NJ (US); Clifton Dalgard, San Francisco, CA (US); Ahmed Mohyeldin, Washington, DC (US); Ronald Zenk, Eden Prairie, MN (US)

Correspondence Address: MORGAN LEWIS & BOCKIUS LLP, 1111 PENNSYLVANIA AVENUE NW, WASHINGTON, DC, 20004, US

(21) Appl. No.: 10/578,438

(22) PCT Filed: Nov. 8, 2004
(86) PCT No.: PCT/US04/37045

§ 371 Date: Mar. 21, 2007

Related U.S. Application Data
(60) Provisional application No. 60/517,918, filed on Nov. 7, 2003.

(51) Int. Cl. A61K 31/19; A61K 31/21; C12N 15/01

(52) U.S. Cl. 514/506; 435/441; 514/557

(57) ABSTRACT

The present invention relates to the elucidation of specific molecular features of endogenous 2-oxoacid molecules and their derivatives for activating hypoxia-inducible gene expression by inactivating hypoxia-inducible factor hydroxylating enzymes. This invention identifies agents that can be used to induce tissue vascularization, treat anemias, induce tolearance to stroke and heart attacks, improve tissue healing and improve organ transplantation.

### GOVERNMENT SUPPORT

NIH grant NS37814 and Department of Defense grant MDA 905-92-Z-0003

### FIELD OF THE INVENTION

The invention relates generally to the changes in gene expression in human tissues, which bring about improved survival in conditions of reduced blood flow and oxygen supply. The invention relates specifically to the pharmacological activation of hypoxia-inducible gene expression by 2-oxoacids and their derivatives. This application is related to U.S. Provisional Application 60/517,918 which is herein incorporated by reference in its entirety.

### BACKGROUND OF THE INVENTION

The ability to adapt to low oxygen levels, perhaps best known in the context of acclimation to high altitudes, is crucial for survival. Cells adapt to low oxygen by turning on genes that encode for proteins which promote better oxygen delivery via new red blood cell synthesis (erythropoiesis) and development of new blood vessels (angiogenesis). Other hypoxia-stimulated gene products stimulate glucose uptake, enhance anaerobic glucose metabolism, and induce several cell survival mechanisms (table 1). Athletes have long capitalized on such hypoxic adaptations to improve their physiological performance. In addition, the deliberate adaptation of cells to sublethal hypoxia has also been shown to reduce tissue injury from strokes and heart attacks. The hypoxic challenge in these settings, referred to as hypoxic preconditioning, has been shown in many animal studies to constitute one of the most potent strategies in reducing ischemic injury. Hypoxic preconditioning mediated protection against ischemic injury has been shown to occur in vivo in a variety of organ systems, including the heart brain, spinal cord, retina, liver, lung and skeletal muscle (Hawaleshka et al. (1998) Can. J. Anaesth. 45, 670-82). Ischemic or hypoxic preconditioning is also useful in prolonging the survival and grafting efficiency of donated tissue used for transplants.

The mechanisms by which hypoxia induces the expression of survival promoting genes are rapidly becoming clarified. Hypoxia (oxygen levels below 5%) regulates gene expression predominantly via the transcription factor HIF-1 (hypoxia-inducible factor-1) (Semenza (2001) Trends Mol. Med. 7, 345-350). Two different proteins called HIF-1 alpha (HIF-1a) and HIF-1 beta (HIF-1b) make up this transcription factor and the level of the HIF-1a component is specifically regulated by oxygen tensions. The regulation of HIF-1a levels involves a novel oxygen sensing mechanism which directly controls the degradation of the HIF-1a protein (FIG. 1). Both HIF-1a and HIF-1b are constitutively synthesized in most cells of the body. However the HIF-1a protein is continuously degraded in the presence of oxygen. A newly discovered family of enzymes known as HIF-1-alpha-prolyl hydroxylases regulate the oxygen-dependent degradation of HIF-1a. These enzymes catalyze the oxygen-dependent hydroxylation of a key proline residue in the HIF-1a protein. This modification, in turn, directs the ubiquitination and proteasomal degradation of the HIF-1 protein. Another recently identified HIF-1a asparagine hydroxylase enzymatic activity also appears to be involved in inhibiting the trascriptional activation ability of HIF-1 under normal oxygen tensions. The HIF-1a asparagine hydroxylases has been termed Factor Inhibiting HIF or HIF-1 by other investigators. All of the HPH and FIH-1 require several co-factors for their activity: oxygen, iron, ascorbate, and 2-oxoglutarate (FIG. 2). In the absence of oxygen therefore, HIF-1a is not hydroxylated or degraded, and as a result, its concentration increases dramatically (Semenza (2001) Trends Mol. Med. 7, 345-350). This allows the HIF-1a and beta subunits to dimerize, translocate to the nucleus and activate the transcription of several genes that promote survival under low oxygen levels (FIG. 1). The discovery of the HPH enzyme mechanism also explains why iron chelators such as desferrioxamine (DFO) can activate HIF-1 and turn on genes similar to those induced by hypoxia. Cobalt and nickel salts, which presumably compete for the iron sites in HPH also mimic hypoxia in regulating HIF-1 and hypoxic gene expression. Both DFO and cobalt have been used effectively to perform hypoxic preconditioning mediated cell protection in animal models of disease (Jones et al. (2001) J. Cereb. Blood Flow Metab. 21, 1105-1114). Although the toxicity of these agents precludes their use in humans, their ability to induce protective preconditioning similar to that seen with hypoxia demonstrates that the pharmacological manipulation of HIF-1a levels by means other than hypoxia is a powerful therapeutic strategy. Recently, molecular interactions at the other cofactor sites have also been shown to regulate HPH activity, HIF-1a levels, and the expression of hypoxia-inducible genes. Thus, artificial analogs of 2-oxoglutarate, such as N-oxalylglycine (NOG) or the cell permeant dimethyloxalylglycine (DMOG), have been shown to block the activity of the HPHs and FIH-1 and thus allow activation of HIF mediated gene expression (Warnecke et al. (2003) FASEB J. 17, 1186-1188). However, these artificial 2-oxoglutarate analogs are not specific in inhibiting HPHs or FIH-1 as they were initially designed to inhibit procollagen proline hydroxylases, the enzymes involved in collagen synthesis.

The HPH, FIH-1, and procollagen proline hydroxylases all belong to the large class of enzymes know as iron and 2-oxoglutarate dependent dioxygenases. These enzymes occur widely in nature and perform valuable biological hydroxylations (Hanauske-Abel et al. (2003) Curr. Med. Chem. 10, 1005-1019). The reaction cycle for these enzymes is depicted in FIG. 2. One peculiarity of these enzymes is that they are syn-catalytically inactivated. This means that as a result of catalyzing iron mediated oxidations, these enzymes either become oxidized at critical amino acid residues or the redox state of the iron becomes useless in carrying out sustained reaction cycles. This syn-catalytic inactivation can be prevented and or reversed by ascorbate (FIG. 2). Many cell lines have recently been shown to express significant HIF-1a protein levels and HIF-mediated gene expression in the absence of hypoxia and this is reversible by ascorbate (Knowles et al. (2003) Cancer Res. 63, 1764-1768). This suggests that, in many cells, HPHs and FIH-1 may exist in an inactivated form or may be made inactive by some mechanism that is ascorbate reversible. So far, no clear understanding of this phenomenon has been achieved and no pharmaceutical approach has been developed to take advantage of a potential HPH and FIH-1 inactivating mechanism.

Certain pharmacological agents such as iron chelators, iron displacing metals, or 2-oxoglutarate antagonists such as NOG or DMOG are general inhibitors of the 2-oxoglutarate dependent enzymes. This family of enzymes is also differentially sensitive to a variety of naturally occuring 2-oxoacids and their derivatives (Hanauske-Abel et al. (2003) Curr. Med. Chem. 10, 1005-1019, Sze-Fong Ng et al. (1991) J. Biol. Chem. 266, 1526-1533, Kaule et al. (1998) Matrix Biol. 17, 205-212). Thus, pyruvate does not inhibit the collagen synthesizing enzymes in humans (Cerbon-Ambriz et al. (1987) Lab Invest. 57, 392-396) but does inhibit such enzymes in certain underwater dwelling worms (Kaule et al. (1998) Matrix Biol. 17, 205-212). Although 2-oxoglutarate derived inhibitors that were developed for the inhibition of collagen synthesis do inhibit HPHs and FIH-1, the specific chemical requirements for 2-oxoacid molecules that inhibit HPHs and FIH-1 have not yet been elucidated. Glucose metabolism generates 2-oxoacids, such as pyruvate and oxaloacetate, that are structurally related to 2-oxoglutarate (FIG. 3). Amino acid metabolism also generates branched chain 2-oxoacids structurally resembling 2-oxoglutarate. It is possible that these naturally occuring 2-oxoacids are biological regulators of HPHs and FIH-1. It is also possible that these agents and their derivatives may be used to develop novel pharmaceutical agents to regulate hypoxic gene expression.

### SUMMARY OF THE INVENTION

The present invention relates to the elucidation of specific molecular features of endogenous 2-oxoacid molecules and their derivatives for activating hypoxia-inducible gene expression by inactivating hypoxia-inducible factor hydroxylating enzymes. This invention identifies agents that can be used to induce tissue vascularization, treat anemias, induce tolerance to stroke and heart attacks, improve tissue healing, protecting against radiation injury, improving immune function and improve organ transplantation.

An embodiment of the present invention relates to a method for activating HIF-1a mediated gene expression in a cell, comprising administering to said cell a composition comprising at least one 2-oxoacid selected from the group consisting of alpha-ketoisovalerate, alpha-ketoisocaproate, alpha-keto-beta-methylvalerate, oxaloacetate, methyl esters thereof, ethyl esters thereof, glycerol esters thereof and butandiol dipyruvate. In one embodiment, said HIF-1a mediated gene expression includes activation of expression of at least one gene selected from the group consisting of genes encoding vascular endothelial growth factor (VEGF), glucose transporter isoform 3 (Glut-3), aldolase A (aldo A) and erythropoietin. In another embodiment, said 2-oxoacid inhibits hydroxylation of HIF-1a in said cell. In a further embodiment, said hydroxylation is mediated by a prolyl hydroxylase or an asparagine hydroxylase.

One more embodiment of the present invention relates to a method for inducing hypoxic adaptation in a mammal in need of such adaptation, comprising administering to said mammal a composition comprising at least one 2-oxoacid selected from the group consisting of pyruvate, oxaloacetate, alpha-ketoisovalerate, alpha-ketoisocaproate, alpha-keto-beta-methylvalerate, methyl esters thereof, ethyl esters thereof and glycerol esters thereof. In another embodiment of this invention, said hypoxic adaptation is induced in a human who is at risk of heart attack, stroke or pregnancy-associated eclampsia. In a further embodiment, said hypoxic adaptation is induced in a human suffering from asthma, diabetes, epilepsy, anemia or cardiac arrythmias. In another embodiment, said hypoxic adaptation is induced in a human who has been exposed to high altitude or smoke inhalation.

A further embodiment of this invention relates to a method of promoting tissue neovascularization in a mammal comprising administering to said patient a composition comprising at least one 2-oxoacid selected from the group consisting of pyruvate, oxaloacetate, alpha-ketoisovalerate, alpha-ketoisocaproate, alpha-keto-beta-methylvalerate, oxaloacetate, methyl esters thereof, ethyl esters thereof and glycerol esters thereof. In one embodiment of this invention said tissue vascularization is promoted in a human who has a peripheral vascular disease selected from the group consisting of atherosclerosis, vasculitis, phlebitis and thrombosis. In another embodiment of this invention said tissue vascularization is promoted in a human who is in need of wound- or bum-healing.

Another embodiment of this invention relates to a method for accelerating the development of proper oxygen homeostasis in a fetus comprising administering to a pregnant human a composition comprising at least one 2-oxoacid selected from the group consisting of pyruvate, oxaloacetate, alpha-ketoisovalerate, alpha-ketoisocaproate, alpha-keto-beta-methylvalerate, oxaloacetate, methyl esters thereof, ethyl esters thereof and glycerol esters thereof. In one embodiment of this invention, the development of proper oxygen homeostasis in a fetus is accelerated in said pregnant human is at risk for premature delivery.

A further embodiment of this invention relates to a method for protecting a mammal against radiation injury comprising administering to said mammal a composition comprising at least one 2-oxoacids selected from the group consisting of pyruvate, oxaloacetate, alpha-ketoisovalerate, alpha-ketoisocaproate, alpha-keto-beta-methylvalerate, methyl esters thereof, ethyl esters thereof, and glycerol esters thereof. In one embodiment of this invention, said composition is administered prophylactically, before exposure to radiation, during exposure to radiation or after exposure to radiation.

In all the embodiments of the invention described above said composition is administered by at least one method selected from the group consisting of oral administration, mucosal administration, ocular administration, subcutaneous injection, transdermal administration, and combinations thereof. Generally, the administration of said composition is repeated in time intervals in the range of from about one hour to about forty-eight hours.

### BRIEF DESCRIPTION OF FIGURES

FIG. 1: HIF-1a hydroxylases and the regulation of gene expression by hypoxia (A) HIF-1a protein hydroxylases are the best candidates for oxygen sensors in multicellular organisms to date. These enzymes require 2-oxoglutarate, ascorbate, Oxygen, and iron, thus explaining their inhibition under hypoxia or by iron chelators such as desferrioxamine (DFO) and competing metals such as cobalt. It is not known whether molecular interactions at the other indicated cofactor sites can regulate the activities of these enzymes. (B) Regulation of gene expression by hypoxia via HIF-1a protein hydroxylase activity. Two separate activities hydroxylate HIF-1a on distinct proline and asparagine residues to regulate the proteolysis and transactivating activity of HIF-1 respectively. These activities are inhibited wider hypoxia allowing HIF-1a to accumulate and for the HIF-1 complex to activate gene expression (dashed lines). Abbreviations: DFO=desferrioximine, 2-OG=2-oxoglutarate, Asc=ascorbate; bHLH=beta-helix-loop-helix domain, PAS=Per-Arnt-Sim domain, C-TAD=c-terminal transactivation domain, ODD=oxygen-dependent degradation domain, pVHL=von Hippel-Lindau protein, HIF-b=beta subunit of HIF, HRE=HIF regulatory element.

FIG. 2: Putative enzymatic cycle for HIF prolyl hydroxylases HPH and FIH-1 are members of the 2-oxoglutarate dependent dioxygenase enzyme family. These enzymes require iron, 2-oxoglutarate, and oxygen to carry out biological hydroxylations. This figure depicts a putative sequence of events that has been proposed for many members of this enzyme family (Hanauske-Abel et al. (2003) Curr. Med. Chem. 10, 1005-1019). (A) HPH (grey C-shaped structure) bind iron (Fe). (B) The HPH-iron complex binds 2-oxoglutarate. The 2-oxo group coordinates with iron while the 5-carbon end of the molecule interacts with a different site. (C) This complex allows one atom of molecular oxygen to be inserted into the 2-oxoglutarte molecule to yield succinate and carbon dioxide while the other oxygen atom forms a complex with the enzyme-bound iron. (D) The iron-complexed oxygen is used to hydroxylate proline 564 within the HIF-1a oxygen dependent degradation domain. HPHs also carry out similar hydroxylation on proline 402 while the FIH-1 enzyme hydroxylates asparagine 803. (E) Most enzymes that utilize this mechanism of hydroxylation become syncatalytically inactivated over time. This inactivation may involve redox reactions between oxygen, iron, and the enzyme and can be favored by certain conditions such as the presence of a pseudo-substrate. (F) Enzymes inactivated in this way can be re-activated with ascorbate which appears to bind these enzymes in a manner similar to 2-oxoglutarate.

FIG. 3: Glucose metabolism and HIF-1 regulation (A) Abbreviated scheme of glycolysis and strategy for determining the key glucose metabolite responsible for HIF-1a upregulation. During glycolysis, glucose is sequentially metabolized to pyruvate, which can then enter mitochondria for further metabolism or can be converted into lactate. Complex interconversions also link pyruvate and oxaloacetate (OAA) levels. The glucose analog 2-deoxyglucose (2DG) can only proceed to 2-deoxyglucose 6 phosphate and cannot be further metabolized. Glyceraldehyde 3 phosphate dehydrogenase (GAPDH) is a key enzyme in glycolysis that can be selectively inhibited by iodoacetic acid (IAA). The transport of pyruvate and lactate across cellular membranes occurs through a specific carrier that is blocked by 4-hydroxycinnamate (4-CIN. Thus 4-CIN prevents pyruvate entry into mitochondria. The interconversion of lactate to pyruvate is mediated via lactate dehydrogenase (LDH), which can be selectively blocked by oxamate. Use of the various inhibitors and intermediates shown here allowed us to determine which key metabolite was responsible for HIF-1a activation. (B) Structural comparisons of 2-oxoglutarate (2-OG), succinate (Succ), oxaloacetate (OAA), and pyruvate (Pyr). 2-OG, OAA and Pyr are all 2-oxoacids based on the keto group at position 2, while succinate is not.

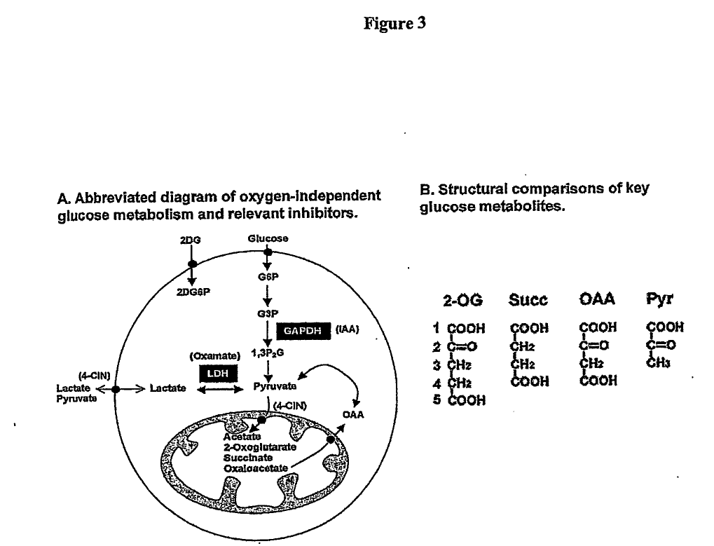

FIG. 4: Regulation of HIF-1a levels by glucose metabolism Western blots are shown in which nuclear extracts of U87 glioma cells were probed with a specific monoclonal antibody recognizing the HIF-1a protein. (A) U87 glioma cells cultured in DMEM were switched to Krebs buffer containing 5.5 mM glucose (Glc) and then evaluated for nuclear HIF-1a levels at various times by western blot analysis. (B) HIF-1a levels were measured after four hours incubation of cells in Krebs buffer containing the indicated glucose concentrations or with 5.5 mM 2-deoxyglucose (2-DG) substituted for glucose. (C) HIF-1a levels were measured in U87 cells cultured in glucose-free Krebs buffer following treatment for four hours under normoxia (21% oxygen) hypoxia (1% oxygen) or with 150 μm desferrioxamine (DFO). (D) Induction of HIF-1a by glucose was monitored in the presence of 50 μM IAA or 1 mM 4-CIN. (E) HIF-1a levels were measured in U87 cells cultured for four hours in Krebs in which glucose was replaced with 3 mM concentrations of lactate (Lac), pyruvate (Pyr), citrate (Cit), 2-oxoglutarate (2-OG), succinate (Succ), or alanine (Ala). Results are representative of experiments repeated at least three times. This figure demonstrates that HIF-1a levels can be regulated by glycolytic metabolites and that the mechanism involved is non-obvious and distinct from that involving hypoxia

FIG. 5: Regulation of HIF-1a protein levels by lactate and pyruvate U87 cells were maintained in MEM overnight (A) The production of lactate in the culture buffer was measured over time following change from MEM to 5.5 mM glucose-containing Krebs buffer. Similar measurements were made in the presence of 50 μM IAA or in glucose-free Krebs buffer. (B) Buffer lactate and pyruvate levels were measured following four hour culture in 5.5 mM glucose containing Krebs buffer alone (open bars) or in the presence of 10 mM oxamate (closed bars) (C) Nuclear HIF-1a protein levels were determined four hours following switching of cells from MEM (control, CT) to Krebs containing either 0.55 mM glucose, or glucose replaced with the indicated concentrations of lactate or pyruvate. (D) HIF-1a levels were measured after switching cells from MEM to glucose-free Krebs containing 2 mM lactate or pyruvate. HIF-1a induction by four hours treatment with 5.5 mM glucose-containing Krebs (Glc) is shown for comparison. (E) Digitonin-permeabilized cells were treated with 1% oxygen or Krebs containing either 5.5 mM glucose or 2 mM pyruvate in the presence of 50 μM IAA. Permeabilized cell in lanes 5 and 6 were treated with 3 mM NAD or NADH respectively in glucose free Krebs. Nuclear HIF-1a levels were determined four hours later. (F) HIF-1a levels were determined after four hour treatment of cells in glucose-free Krebs (lane 1) or 5.5 mM Glucose containing Krebs. Glucose induced HIF-1a in both permeabilized or intact cells and neither NAD or NADH (3 mM each) had any effect on this induction. Catalase (1000 and 2000 units/ml) also had no effect. (G) HIF-1a levels were determined after four hours treatment in glucose-free Krebs containing 2 mM lactate or pyruvate with or without 10 mM oxamate. (H) To measure the decay of the HIF-1a protein, HIF-1a measurements were made after four hour treatment under hypoxia (lane 1), four hours hypoxia followed by 30 minutes normoxia (lane 2), four hour treatment with 150 μM DFO (lane 3), four hours DFO followed by addition of 100 μM CHX for one hour (lane 4), four hours treatment with 2 mM pyruvate (lane 5), and four hours pyruvate followed by addition of 100 μM CHX for one hour (lane 6). (H) HIF-1a levels were determined in digitonin-permeabilized cells treated for four hour with Krebs containing no glucose (lane 1), 1 mM pyruvate (lane 2), or 1 mM pyruvate and 10 mM 2-OG (lane 3). Except where indicated, all experiments were carried out under normoxia and were repeated at least three times with-similar results. This figure along with FIG. 3 demonstrates that of all the key changes that take place during glucose metabolism, it is the accumulation of pyruvate that promotes HIF-1a accumulation. Furthermore, pyruvate appears to mediating its actions by stabilizing HIF-1a protein levels.

FIG. 6: Pyruvate analogs and oxaloacetate efficiently enhance HIF-1a protein levels Human U87 glioma cells (panels A-C) and other cell lines (D) were treated with glycolytic and Krebs cycle intermediates as well as ethyl and methyl esters of pyruvate and analyzed for HIF-1a accumulation in nuclear extract. With the exception of lactate, as discussed above, no other glycolytic intermediates were found to activate HIF-1a accumulation besides pyruvate. Of all Krebs cycle, only oxaloacetate (OAA) was able to stimulate HIF-1a accumulation. (B) The effects of pyruvate and OAA were mimicked by ethyl- and methyl-esters of pyruvate and were as pronounced as those of the known 2-oxoglutarate antagonist dimethyloxalylglycine (DMOG). (C) The effects of OAA were as potent as pyruvate and (D) were seen in several other cell lines including U251 human glioma cells, Hep3B human hepatoma cells, and DU145 human prostate carcinoma cells. Hela human cervical carcinoma cells, normal human astrocytes, and normal human prostate epithelium cells also displayed similar responses to pyruvate and oxaloacetate (data not shown).

FIG. 7: Structure-activity requirements for 2-oxoacids that elevate HIF-1a levels HIF-1a protein levels accumulate due to inhibition of HPH activity as a result of either hypoxia, iron removal, or competitive antagonism of 2-oxoglutarate by artificial analogs such as N-oxalylglycine or dimethyloxalylglycine. We have found that naturally occurring 2-oxoacids can promote HIF-1a accumulation and their structural requirements for this activity are shown in this diagram. We determined the ability of each shown structure to induce HIF by exposing digitonin-permeabilized human glioma cells (U87, U251) to 1 mM doses. N-oxalylglycine and its esterified precursor dimethyloxalylglycine consistently enhanced HIF-1a levels. Of all the other compounds shown, pyruvate, oxaloacetate, alpha-ketoisovalerate, alpha-ketoisocaproate, and alpha-keto-beta-methylvalerate (boxed) were the only agents capable of stimulating HIF-1a accumulation. Lactate can also stimulate HIF-1a after its conversion to pyruvate (see FIG. 5). These data establish the necessity of the alpha-keto group. However, the ineffectiveness of alpha-ketobutyrate and alpha-ketoadipate also provide empirical data that other structural features are important.

FIG. 8: HIF activation by 2-oxoacids is independent from energy metabolism (A) U251 glioma cells were cultured in Krebs buffer without glucose or with the indicated additions at 2 mM each. At four hours, ATP levels were measured in cell extracts using the luciferase method. Although small variations in ATP were observed with the various treatments, there was no correlation with respective actions of these agents on HIF-1a accumulation. (B) Direct addition of 1 mM ATP to digitonin permeabilized cells also had no effect on HIF-1a levels.

FIG. 9: Pyruvate stabilizes HIF-1a by acting at a step prior to ubiqitinylation (A) U87 glioma cells were cultured for four hours in glucose free Krebs buffer alone (lane 1) or with the indicated treatments (DFO=100 μM, DMOG=1 mM, Glucose=2 mM, Pyruvate=2 mM, Lactacystin-beta lactone (Lbl)=20 μM. All treatments except the 1% oxygen were performed under 20% oxygen. Whole cell extracts were then prepared and probed for HIF-1a protein levels. Only cells treated with the proteasome inhibitor Lbl, displayed HIF immunoreactivity with the characteristic larger molecular weight smear of ubiquitinylated HIF-1a. (B) U373 glioma cells were treated under conditions similar to those in (A). Oxaloacetate and succinate were used at 2 mM. Note that the ubiquitinylated HIF-1a produced via Lbl treatment does not translocate to the nucleus. Also note the ineffectiveness of succinate.

FIG. 10: Demonstration of HIF-1a activation by branched chain 2-oxoacids U251 cells were treated with 2 mM doses of the indicated 2-oxoacids for four hours in glucose-free Krebs buffer. Cells were then washed, fixed and stained for HIF-1a protein.

FIG. 11: Pyruvate and Oxaloacetate compete for 2-oxoglutarate binding to HIF Prolyl hydroxylases (A) Human glioma cells express HPH homologues 1, 2 and 3. RT-PCR was performed using specific primers to demonstrate the presence of HPH homologues in the glioma cell lines used to gather most of our data. The pattern of expression seen is similar to those of normal human tissues. (B) HPH bind to immobilized 2-oxoglutarate. This assay is a measure of step B in FIG. 2. Epoxy-activated Sepharose beads covalently coupled with 2-oxoglutarate were incubated with in vitro translated ³⁵S-labeled HPH homologues in the presence and absence of 250 mM iron sulfate at room temperature and then pelleted by centrifugation. Following four further washes radiolabel associated with the pellets was measured via scintillation counting. More than 50% of the radiolabel bound was iron dependent (C) Nearly half of the total binding of HPH to the 2-oxoglutarate column could be displaced by 20 mM 2-oxoglutarate but not by 20 mM succinate. (D) Iron dependent HPH binding to immobilized 2-oxoglutarate is displaced by pyruvate (20 mM) and oxaloacetate (20 mM).

FIG. 12: Pyruvate and oxaloacetate do not support hydroxylation of HIF-1a ODD peptide We examined whether pyruvate or oxaloacetate influenced the prolyl hydroxylation of HIF-1a by monitoring the ability of HPH homologues to confer ³⁵S-pVHL binding activity onto a biotinylated 19mer peptide containing the key proline564 residue of the HIF-1a ODD (see FIG. 1). After incubating the peptide with the HPHs and the indicated reagents, ³⁵S-pVHL was added followed by addition of streptavidin-coated beads to pull down the HIF-1a peptide. The reaction was pelleted, the pellet was washed and then solubilized for SDS-PAGE analysis followed by autoradiography to reveal the captured ³⁵S-pVHL. Using in vitro translated HPH homologues we first optimized assay conditions with respect to the required HPH substrates and co-factors. (A) 2-OG was absolutely required for activity as shown by this dose curve. Conditions for other reagents were: ascorbate=2 mM, iron sulfate=250 μM, DTT=1 mM. () Iron was also absolutely required up to a maximal of about 100 μM. Conditions: ascorbate=2 mM, 2-OG=2 mM, DTT=1 mM. (C) Although some activity was seen in its absence, ascorbate dose-dependently enhanced activity. Conditions: 2-OG=125 μM, iron sulfate=250 μM, DTT=1 mM. (D) Under conditions where all other reagents were kept constant as above, 1 mM amounts of Pyr or OAA could not substitute for 100 μM 2-OG in catalyzing proline hydroxylation of HIF-1a peptide by any of the three HPH homologues. In all assays 5 μl (about 20 ng) of enzyme and 1 μg of peptide was utilized.

FIG. 13: In vitro effects of 2-OG analogs on recombinant HPH activity HPH activity was assessed via the ³⁵S-pVHL pulldown assay as in FIG. 11. Activity of in vitro translated HPH homologues was determined in the absence and presence of the 2-OG analogs N-oxalylglycine (NOG), pyruvate or OAA at 1 mM. While inhibition by NOG is clearly evident, the effects of pyruvate and oxaloacetate are less consistent at either 5 mM or 25 mM [2-OG].

FIG. 14: Ascorbate-reversible inhibition of in vitro recombinant HPH activity The ³⁵S-pVHL capture assay used in FIG. 11 was employed to determine whether OAA or Pyr could act as inhibitors of HPH activity. Conditions employed were: 2-OG=100 mM, iron sulfate=20 mM, DTT=1 mM. Ascorbate concentrations were varied as indicated and OAA or Pyr were added where indicated at 1 mM. Both OAA and Pyr appeared to inhibit the HPH-1 and HPH-2 activity with their effects being more apparent at lower ascorbate doses. HPH-3 did not appear to be sensitive to OAA or Pyr.

FIG. 15: Pyruvate or oxaloacetate-induced HIF-1a accumulation is blocked by ascorbate U87 and U251 glioma cells were treated for four hours in glucose-free Krebs buffer under the indicated conditions. Pyruvate and OAA were included at 1 mM where indicated. (A) Nuclear accumulation of HIF-1a in U87 cells was assessed in nuclear extracts via western blotting. (B) Nuclear accumulation of HIF-1a in U251 cells was analyzed by immunohistochemistry. Note the inhibition of HIF-1 accumulation by ascorbate (100 μM under pyruvate and oxaloacetate treatment but not under hypoxia. Also note that 2-oxoglutarate (10 mM) was unable to reverse either inducer. Unstimulated U87 cells are not shown in this figure.

FIG. 16: Ascorbate reverses the prolonged HIF-1a accumulation (A) Comparison of HIF-1a decay in U251 following induction with either hypoxia or pyruvate. U251 cells were cultured in glucose-free Krebs under hypoxia or with 1 mM pyruvate in normoxia for four hours. Following this cells were switched to glucose free Krebs in normoxia and fixed in formaldehyde at the indicated times. Cells were then stained for HIF-1a immunoreactivity. Note the rapid decay of nuclear HIF-1a staining after having been induced by hypoxia versus pyruvate. (B) U87 cell treated for four hours under hypoxia show prominent HIF-1a induction which is completely degraded by thirty minutes of re-oxygenation. U87 cells were treated in glucose-free Krebs buffer with or without 1 mM pyruvate or oxaloacetate. After four hours cells were washed in glucose-free Krebs for various times with or without 100 μM ascorbate being included in the wash Note that the pyruvate and oxaloacetate-induced HIF-1a accumulation persists for a long time after the inducing agents have been washed away. Inclusion of ascorbate in the wash enhanced the rate of HIF-1a decay.

FIG. 17: Inactivation of cellular HPH activity by pyruvate and oxaloacetate U251 cells were cultured for four hours in glucose-free Krebs buffer with or without the indicated additions. Whole cell extracts were then prepared and used as a source of HPH enzyme to hydroxylate a biotinylated peptide from the HIF-1a ODD containing proline 564. Proline hydroxylation was measured by the ability of streptavidin coated beads to pulldown the hydroxyproline ³⁵S-pVHL complex as in FIGS. 12-14. (A) When cells were treated with 1 mM pyruvate or oxaloacetate there was a marked reduction in the HPH activity of U251 extracts. Inclusion of 100 μM ascorbate during the cell incubation prevented this loss of activity. (B) Similar experiments with hypoxia or DMOG showed no such loss of HPH activity.

FIG. 18: 2-oxoacids activate HIF-mediated gene expression in human cell lines Effective gene expression by HIF not only involves HIF protein stabilization via inhibition of HPH enzymes but also HIF-1 binding to DNA, inhibition of FIH-1 activity, and gene transcription (see FIG. 1). (A) Glioma cells used in our studies express FIH-1 as assessed using RT-PCR. (B) nuclear extracts from pyruvate treated U87 cells express binding activity for HRE DNA. (C) U87 cells also upregulate mRNA levels of several gene known to be regulated by HIF, such as vascular endothelial growth factor (VEGF), glucose transporter isoform 3 (Glut-3) and aldolase A (Aldo A). Expression of beta-actin, a housekeeping gene not under HIF regulation is not affected by pyruvate. (D) Human Hep3B hepatoma cells express erythropoietin (epo) mRNA and this expression id dose dependently increased by pyruvate. (E) U373 cell were transfected with a green fluorescent protein (GFP) construct under the control of an HIF regulatory element (HRE) containing promoter and then cultured for eight hours in glucose-free medium under the indicated conditions. GFP (green fluorescence) was expressed when cells were treated with 1% oxygen or DFO. Pyruvate also enhanced GFP expression. (F) HRE regulated luciferase was used to demonstrate activation of HIF regulated genes by 2-oxoacid and their analogs. U251 cells stably transfected with a luciferase construct under the control of an HRE containing promoter were cultured for six hours in glucose free Krebs with the following conditions: 1=control, 2=1% oxygen, 3=pyruvate (2 mM), 4=OAA (2 mM), 5=Ethylpyruvate (2 mM), 6=Glucose (5.5 mM), 7=DMOG (0.5 mM), 8=Lactacystin-beta lactone (20 mM). Note the enhanced HRE-luciferase expression by hypoxia, DFO and 2-oxoacids, but not by lactacystin.

FIG. 19: HIF-mediated gene expression is selectively reversed by ascorbate U251 cells stably expressing HRE-luciferase were cultured in glucose-free Krebs buffer with the indicated conditions for eight hours. Pyruvate, oxaloacetate, and DMOG were added at 1 mM each. Activation of HRE-luciferase by pyruvate or oxaloacetate is distinguished from that by hypoxia or DMOG by its selective reversal by ascorbate. No reversal was seen with 10 mM 2-oxoglutarate.

FIG. 20: 2-Oxoacids activate AM in brain cells (A) Primary cultures of rat cerebral cortical neurons grown in Neurobasal media were treated with either 1% oxygen or 3 mM pyruvate for four hours and then assayed for HIF-1a immunoreactivity. Note the increased nuclear accumulation of HIF-1a by both hypoxia and pyruvate. (B) Similar experiments were carried out with primary cultures of rat astrocytes, except that nuclear extracts were prepared and assayed for HIF-1a by western blotting. (C) Ten day old rats were subjected to hypoxia (8% oxygen) or were injected with 2 g/kg pyruvate i.p. Four hours later, rats were sacrificed, their brains were harvested, and nuclear extracts were prepared. HIF-1a levels were determined by western blotting. (D) In a similar experiment ten day old rats were exposed to 0.1% carbon monoxide to produce systemic hypoxia or were injected i.p. with 2 g/kg OAA.

FIG. 21: Oxaloacetate preconditioning can protect neurons from oxygen glucose deprivation OAA preconditioning involved the addition of OAA at different concentrations directly adding it to the medium 48 hours prior to oxygen-glucose deprivation (OGD). Immediately before starting OGD the Neurobasal medium (N/B27) was removed and washed out with phosphate buffered saline. Thereafter, OGD was induced with Krebs buffer without glucose and cells were placed in hypoxia chamber (1% oxygen) for two hours. In control experiments the medium was replaced by regular glucose containing Krebs buffer and the cells were incubated in a normoxic atmosphere of 20 to 21% oxygen. Immediately after OGD, buffers from different treatment groups were removed and replaced with fresh medium, cells were assayed for cell viability 24 hours post insult with MTT reduction. OAA showed protective effects at concentration of 1 mM and this effect was statistically significant (*p<0.05 at 3 mM oxaloacetate treatments).

### DETAILED DESCRIPTION

The invention is derived from the discovery that certain endogenous 2-oxoacids are responsible for the regulation of HIF-1 levels under normoxic (20 to 21% oxygen) conditions. Specifically, the endogenous 2-oxoacids pyruvate and oxaloacetate compete for the 2-oxoglutarate binding site in HIF hydroxylating enzymes and then lead to their inactivation. This results in long-lasting HIF-1a accumulation and activation of HIF-1a mediated gene expression, even in the presence of oxygen.

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, the term “binding” refers to the adherence of molecules to one another, such as, but not limited to, enzymes to substrates, proteins to proteins, transcription factor proteins to DNA, and DNA or RNA strands to their complementary strands. Binding occurs because the shape and chemical nature of parts of the molecule surfaces are complementary. A common metaphor is the “lock-and-key” used to describe how enzymes interact with their substrate.

As used herein, the term “transcription factor” refers to any protein or protein complex that binds to specific regulatory regions of DNA to stimulate gene expression. Examples include, but are not limited to, the HIF-1a protein.

As used herein, the term “gene expression” refers to the enhanced production of messenger RNA (mRNA) from DNA, which eventually leads to enhanced protein coded for by the mRNA and to enhanced protein function.

As used herein, the term “HIF-1” or “HIF-1 protein” refers to a transcription factor comprising two different proteins called HIF-1 alpha (HIF-1a) and HIF-1 beta (HIF-1b) as previously described (Wang et al. (1995) J. Biol. Chem. 270, 1230-1237; U.S. Pat. Nos. 6,562,799 and 6,222,018) and includes all known isoforms, including those of mammals, especially human HIF-1.

As used herein, the term “hypoxia” refers to oxygen tensions below 5 percent (%). Normal air is composed of 20 to 21 percent oxygen, a condition referred to as “normoxia” in the art

As used herein, the term “therapeutic agent” refers to any composition, which integrates the core chemical structure of a 2-oxoacid such as pyruvate and oxaloacetate which is required for binding to HIF-1a hydroxylating enzymes. Examples include, but are not limited to, methyl-, ethyl-,and glycerol-esters of pyruvate and oxaloacetate, alpha-ketoisovalerate, alpha-ketoisocaproate, alpha-keto-beta-methylvalerate, oxaloacetate, methyl esters thereof, ethyl esters thereof and glycerol esters thereof. Other examples may include agents that raise pyruvate and oxaloacetate tissue levels by preventing their breakdown.

Methods of Use

Induction of hypoxic adaptation in heart attack or stroke prone or post-heart attack and post-stroke victims. These two conditions are among the leading causes of death and disability in our society. The few medications available for prevention of heart attacks and strokes today include antihypertensive agents, aspirin and other anti-platelet agents and cholesterol lowering drugs. In animal experiments, hypoxic or ischemic preconditioning provides far more prophylactic protection against heart attack and stroke than all of these other approaches. Furthermore, since the pharmacological induction of hypoxia-activated genes represents a novel approach and a distinct mechanism for providing protection against ischemic insults for which there are no competing products. Such an approach would compliment all preexisting approaches. Our approach would also be essential for improving recovery from such insults. The invention therefore encompasses methods for induction of hypoxic adaptation in heart attack or stroke prone or post-heart attack and post-stroke victims comprising administering one or more therapeutic agents described herein, either alone or in combination.

Preventive treatment to reduce risk in settings of predictable stroke: cardiac bypass surgery, carotid endarterectomy, deep sea diving. In addition to the prophylactic use of 2-oxoacids and their derivatives in protecting against recurrent strokes or heart attacks, the invention can also be utilized for the induction of prophylactic neuroprotection in settings where there is a significant risk of suffering from a stroke. Thus, individuals who undergo cardiac bypass surgery or carotid endarterectomy, two of the most common surgical procedures today, suffer a significant incedence of ischemic brain injury. The invention therefore encompasses methods of preloading these patients with 2-oxoacids and derivatives thereof that induce hypoxia-regulated genes provides significant protection.

Improvement of glucose metabolism in diabetes. Diabetes also continues to be one of the major medical problems facing our society. Type 2 diabetes continues to increase in incidence high blood glucose is also a risk factor for many other diseases. Nearly half of the three dozen or so genes found to be regulated by HIF-1a so far are concerned with enhancing glucose metabolism. This includes not only the uptake of glucose but also its metabolism via key regulatory enzymes. Currently there is no effective clinical strategy for improving glucose metabolism in diabetic patients and treatment is limited to the use of agents that either enhance insulin secretion or enhance insulin receptor sensitivity. The invention therefore encompasses the use of 2-oxoacids and their derivatives to upregulate the expression of glucose transporters and glycolytic enzymes in diabetic patients. Such an approach would also compliment all preexisting approaches and therefore can be used in combination with existing diabetic therapies.

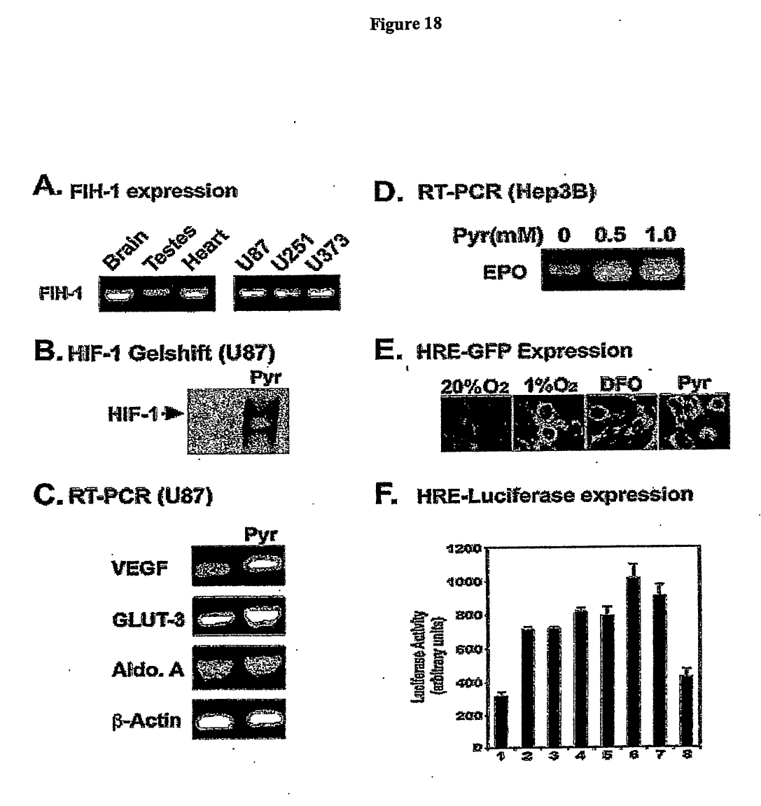

Neovascularization of ischemic tissue in any form of vascular disease. Recovery from stroke and heart attack may require tissue neovascularization, this may also be the case in many peripheral vascular diseases such as atherosclerosis, vasculitis, phlebitis, or thrombosis. Currently there is no routine approach to pharmacologically revascularize issue. Gene therapy approaches that aim to boost tissue levels of vascular endothelial growth factor (VEGF) or fibroblast growth factor 2 (FGF2) are the primary competing technologies, but these have not yet been effectively realized. In fact, the ability of enhanced glycolytic metabolism, and of lactate and pyruvate in particular, to induce the elaboration of angiogenic factors and to enhance angiogenesis has been known for over fifteen years (Jensen et al. (1986) Lab Invest. 54, 574-578). This powerful effect of 2-oxoacids was recently shown in animal models to produce prominant neovascularization (Lee et al. (2001) Cancer Res. 61, 3290-3293). Despite these long-standing observations, the inventors were the first to elucidate the mechanism underlying this phenomenon as the expression of VEGF as well as the VEGF receptor is regulated by HIF-1 (table 1; FIG. 18C).

Improvement of wound and burn healing. Tissue neovascularization and tissue growth is crucial for the healing of wounds and burns. By activating HIF-1a, the topical application of 2-oxoacids could induce the expression of genes that promote angiogenesis and enhance the growth of connective tissue elements and of epithelial cells. Indeed, hypoxia-regulated gene expression plays a prominant in fetal wound regeneration and adult wound repair (Albina et al. (2001) Am. J. Physiol. Cell Physiol. 281, C1971-1977, Scheid et al. (2000) Pediatr. Surg. Int. 16, 232-236). The activation of HIF-1a represents the key event in turning on these genes. The invention therefore encompasses the use of 2-oxoacids such as pyruvate, oxaloacetate, or derivatives thereof incorporated in bandages and applied topically to promote wound and bum healing via HIF-1a activation.

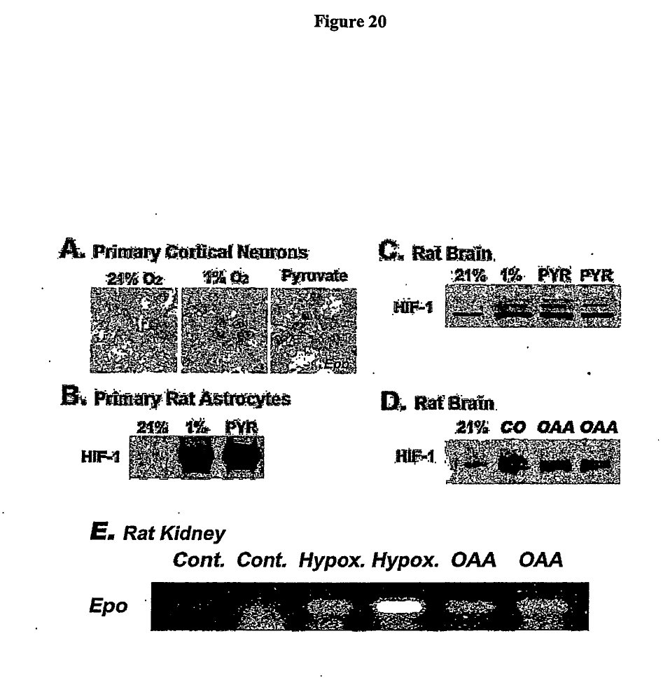

Treatment of anemias. HIF-1a was originally discovered as the transcription factor regulating the expression of the erythropoietin gene. Erythropoietin (EPO) is known to be produced by kidney and liver tissue in response to hypoxia. EPO acts upon the EPO receptor (EPOR) on red blood cell precursors in the bone marrow to bring about a proliferation of red blood cells. EPO is so effective in improving clinical anemias that it is now used routinely in treating a variety of anemias seen in clinical settings. In addition to inducing endogenous EPO production, HIF-1a also induces expression of the transferrin and transferrin receptor genes, which make it possible for red blood cell precursors to turn into mature red blood cells capable of carrying oxygen. The invention therefore encompasses methods of administering therapeutic agents such as the 2-oxoacids, pyruvate and oxaloacetate and derivatives thereof to improve anemia via HIF-1a activation (see FIGS. 18D and 20E). Since oral ingestion of the 2-oxoacids pyruvate or oxaloacetate is harmless to humans, this approach can be readily employed in humans. Furthermore, the effectiveness of these agents can be tested by measuring blood hematocrit levels following administration.

Acclimation to high altitudes. High altitudes atmospheres have the same percent composition of oxygen as low altitudes. However, due to the lower pressure, high altitude air has fewer gas molecules overall and thus lower oxygen levels. Symptoms of high altitude sickness such as headaches, hyperventilation, fatigue and death are due to insufficient oxygen delivery to tissues. Thus insufficient oxygen at high altitudes requires that mammals adapt their physiology in order to survive. The acclimation of manuals to high altitudes is primarily governed by an acute increase in ventilation as well as a sustained increase in HIF-1a mediated gene expression (Semenza (2001) Trends Mol. Med. 7, 345-350). Such genes facilitate mammalian physiology at high altitudes by improving blood oxygen carrying capacity and tissue oxygen delivery while simultaneously improving the oxygen-independent glucose metabolism of the body's cells. The major approach currently available for effectively enhancing adaptation of humans to low oxygen is to ascend slowly thus allowing HIF-1a mediated gene expression to ensue. The invention provides an alternative to this approach in that it encompasses the prophylactic use of therapeutic agents defined herein (i.e., pyruvate or oxaloacetate and their derivatives) to improve and facilitate high altitude acclimation. This application has significant utility amongst travelers that visit high altitudes or military or other personnel that may need to rapidly ascent into areas of low oxygen levels.

Smoke inhalation prophylaxis. Although firefighters do not encounter high altitudes routinely, they are at risk for acute bouts of unexpected hypoxia due to smoke inhalation and carbon monoxide toxicity. Prophylaxis with pyruvate or oxaloacetate or derivatives thereof may markedly reduce the chances of such individuals suffering hypoxic injury and is therefore encompassed in the invention. This approach can also be used to treat patient exhibiting symptoms associated with chronic smoking (e.g., emphysema and related disorders).

Asthma, seizure and cardiac arrythmia prophylaxis. As in induction of hypoxic adaptation in heart attack or stroke, patients with asthma, epilepsy, or cardiac arrythmias are at risk for acute bouts of tissue hypoxia. These conditions can potentially lead to significant hypoxic or anoxic injury. The invention therefore encompasses prophylaxis with pyruvate or oxaloacetate or derivatives thereof to reduce the chances of such individuals suffering hypoxic injury.

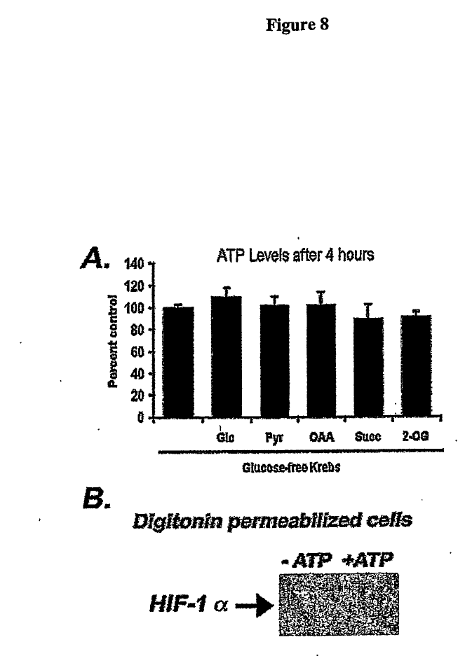

Athletic performance improvement. No population has capitalized upon HIF-1a mediated gene expression more that athletes who compete in highly demanding aerobic sports. In fact, every country's Olympic training centers are located at high altitudes to take advantage of the improvements in physiology that are induced by hypoxia. The hypoxia induced physiological changes described above allow more efficient use of oxygen and also allow the exercising body to utilize anaerobic fuels more efficiently. The result is greater endurance during demanding aerobic exercise or competition. Pyruvate has been used by athletes for a long time during aerobic exercise with the belief that this may provide more metabolic fuel to enhance performance. However, the previously unknown insights offered by our findings (see FIG. 8) suggest that this strategy is flawed. Highly aerobic exercise results in acute tissue hypoxia due to an enhanced demand for oxygen in the face of unchanging supply. Indeed, pyruvate and lactate accumulate significantly during exercise due to their inadequate metabolism by oxygen-requiring reactions. Thus there is plenty of pyruvate accumulated during an acute bout of highly demanding exercise. Oxygen, however, and not pyruvate requires replenishing in these individuals. Alternatively the changes in cell metabolism and improvements in tissue function induced by chronic hypoxia actually lower the tissue demand for oxygen. In addition, the improved oxygen carrying capacity of the blood and improved tissue blood capillary density provoked by hypoxia may be significant factors in improving athletic performance. These physiological changes take days or weeks to express themselves and are initiated via HIF-1a regulated gene expression (Semenza (2001) Trends Mol. Med. 7, 345-350). Thus, effective use of pyruvate and or oxaloacetate for improving athletic performance should focus not on their obvious role as fuel sources but rather on our discovery of their non-obvious role as HIF-1a activators. Thus, the invention encompasses adjustment of chronic ingestion of pyruvate, oxaloacetate, or derivatives thereof by athletes during training and prior to competition to maximize long term changes in HIF-1a mediated gene expression. The increasing clandestine use of EPO by athletes who wish to improve their athletic performance suggests that there is a potentially large market for use of the safer 2-oxoacid derivatives for improving athletic performance.

Improving survival of prematurely born infants. Premature birth has a high degree of association with many diseases in subsequent adult life. The development of proper oxygen homeostasis is crucial for life and activation of HIF-1a is crucial for this to happen (Hawaleshka et al. (1998) Can. J. Anaesth. 45, 670-82). Indeed, HIF-1a knock out mice die in utero. The invention therefore encompasses administration of pyruvate, oxaloacetate or derivatives thereof to expectant mother at high risk for premature delivery may induce HIF-1a in fetal tissues and accelerate the development of proper oxygen homeostasis. This approach is also beneficial in preventing stroke-like episodes from pregnancy-associated eclampsia.

Preservation of donor organs prior to transplant The invention encompasses the use of pyruvate, oxaloacetate or derivatives thereof to induce HIF-1a in the organs of tissue donors prior to harvesting and also the addition of these agents to donated organ storage solution to improve the hypoxic survival of organs during the time that they are not adequately perfused.

Improvement of immune function. Immune activity has recently been shown to be dramatically reduced upon knockout of the HIF-1a gene (Cramer et al. (2003) Cell Cycle 2, 192-193). The invention therefore encompasses the administration of pyruvate and oxaloacetate to immunodeficient individuals to improve outcome form a variety of immunodeficient diseases, including but not limited to, AIDS and exposure to ionizing radiation.

Inhibiting post-radiation HIF-1a activation significantly increases tumor radiosensitivity as a result of enhanced vascular destruction. The present invention therefore relates to the administration of a composition for the prophylactic protection or therapeutic treatment of a subject against radiation injury. In one embodiment of the invention, a therapeutic composition is administered to the subject wherein such composition comprises of compounds that enhance HIF-1a activation in vivo. As a result of the enhanced secretion of the angiogenesis regulator, vascular radiation is diminished owing to the secretion of cytokines which include but not limited to vascular endothelial growth factor (VEGF). Preferably, such compounds are a class of 2-oxoacids described herein.

The route of administration can be any of the commonly accepted practices for the administration of pharmaceutical preparations including, but not exclusively, mucosal administration, oral consumption, ocular administration, subcutaneous injection, transdermal administration, etc. Oral administration is generally preferred.

Mucosal administration of the composition includes such routes as buccal, endotracheal, nasal, pharyngeal, rectal, sublingual, vaginal, etc. For administration through the buccal/endotracheal/pharyngeal/sublingual mucosal, the composition may be formulated as an emulsion, gum, lozenge, spray, tablet or an inclusion complex such as cyclodextrin inclusion complexes. Nasal administration is conveniently conducted through the use of a sniffing powder or nasal spray. For rectal and vaginal administration the composition may be formulated as a cream, douche, enema or suppository.

Oral consumption of the composition may be effected by incorporating the composition into a food or drink, or formulating the composition into a chewable or swallowable tablet or capsule. Ocular administration may be effected by incorporating the composition into a solution or suspension adapted for ocular application such as drops or sprays. Subcutaneous administration involves incorporating the composition into a pharmaceutically acceptable and injectable carrier. For transdermal administration, the composition may be conveniently incorporated into a lipophilic carrier and formulated as a topical creme or adhesive patch. Polylactide fiber, such as that found in sutures that are self-dissolving can generate lactate, which can also subsequently be metabolized by tissues to form pyruvate. This or other suture fabrications can be used for long term local delivery of 2-oxoacids.

The range of dosages and dose rates effective for achieving the desired protection against radiation injury may be determined in accordance with standard industry practices. Preferred dose and dose rate is sufficient composition to provide about 10 to 3,000 mg of 2-oxoacids per day administered once (i.e., each morning), twice (i.e., each morning and evening) or thrice (i.e., with each meal) daily.

Methods to Identify HIF-1 Binding Partners

Another embodiment of the present invention provides methods for use in isolating and identifying binding partners of HIF-1a or HIF-2b. In general, HIF-1a or HIF-1b protein is mixed with a potential binding partner or an extract or fraction of a cell under conditions that allow the association of potential binding partners with HIF-1a protein. After mixing, peptides, polypeptides, proteins or other molecules (e.g., cyteine or histidine) that have become associated with a protein of the invention are separated from the mixture. The binding partner that bound to the protein of the invention can then be removed and further analyzed. To identify and isolate a binding partner, the HIF-1a entire protein can be used. Alternatively, a fragment of the protein can be used.

As used herein, a cellular extract refers to a preparation or fraction that is made from a lysed or disrupted cell. The preferred source of cellular extracts will be cells derived from human skin tissue or the human respiratory tract or cells derived from a biopsy sample of human lung tissue in patients with allergic hypersensitivity. Alternately, cellular extracts may be prepared from normal tissue or available cell lines, particularly cancer cell lines, including glioma cell lines.

A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with the protein of the invention under conditions in which association of the HIF-1a protein with the binding partner can occur. A variety of conditions can be used, the most preferred being conditions that closely resemble conditions found in the cytoplasm of a human cell. Features such as osmolarity, pH, temperature, and the concentration of cellular extract used, can be varied to optimize the association of the protein with the binding partner.

After mixing under appropriate conditions, the bound complex is separated from the mixture. A variety of techniques can be utilized to separate the mixture. For example, antibodies specific to a protein of the invention can be used to immunoprecipitate the binding partner complex. Alternatively, standard chemical separation techniques such as chromatography and density/sediment centrifugation can be used.

After removal of non-associated cellular constituents found in the extract, the binding partner can be dissociated from the complex using conventional methods. For example, dissociation can be accomplished by altering the salt concentration or pH of the mixture. To aid in separating associated binding partner pairs from the mixed extract, the protein of the invention can be immobilized on a solid support. For example, the protein can be attached to a nitrocellulose matrix or acrylic beads. Attachment of the protein to a solid support aids in separating peptide/binding partner pairs from other constituents found in the extract The identified binding partners can be either a single protein or a complex made up of two or more proteins. Alternatively, binding partners may be identified using a Far-Western assay according to the procedures of Takayama et al. (1997) Methods Mol. Biol. 69, 171-184 or Sauder et al. (1996) J. Gen. Virol. 77, 991-996 or identified through the use of epitope tagged proteins or GST fusion proteins.

Alternatively, the nucleic acid molecules encoding HIF-1 can be used in a yeast two-hybrid system. The yeast two-hybrid system has been used to identify other protein partner pairs and can readily be adapted to employ the nucleic acid molecules herein described.

Methods to Identify Agents that Modulate HIF-1 Expression

In one embodiment of the present invention, methods are provided for identifying agents that modulate the expression of a nucleic acid encoding a HIF-1 a or HIF-1b protein. Such assays may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell. Examples of agents which up-regulate the expression of HIF-1a protein include, but are not limited to, 2-oxoacids such as pyruvate, oxaloacetate and derivatives thereof.

In one assay format, cell lines that contain reporter gene fusions between the open reading frame of the HIF-1a gene, or the 5′ and/or 3′ regulatory elements and any assayable fusion partner may be prepared. Numerous assayable fusion partners are known and readily available including the firefly luciferase gene and the gene encoding chloramphenicol acetyltransferase (Alam et al. (1990) Anal. Biochem. 188, 245-254). Cell lines containing the reporter gene fusions are then exposed to the agent to be tested under appropriate conditions and time. Differential expression of the reporter gene between samples exposed to the agent and control samples identifies agents that modulate the expression of a nucleic acid encoding a HIF-1a protein.

Additional assay formats may be used to monitor the ability of the agent to modulate the expression of a nucleic acid encoding a HIF-1a protein. For instance, mRNA expression may be monitored directly by hybridization to the nucleic acids of the invention. Cell lines are exposed to the agent to be tested under appropriate conditions and time and total RNA or mRNA is isolated by standard procedures such those disclosed in Sambrook et al. (2001) Molecular Cloning—A Laboratory Manual, Cold Spring Harbor Laboratory Press).

Probes to detect differences in RNA expression levels between cells exposed to the agent and control cells may be prepared from the nucleic acids encoding a HIF-1a protein. It is preferable, but not necessary, to design probes which specifically hybridize only with target nucleic acids under conditions of high stringency. Only highly complementary nucleic acid hybrids form under conditions of high stringency. Accordingly, the stringency of the assay conditions determines the amount of complementation that should exist between two nucleic acid strands in order to form a hybrid. Stringency should be chosen to maximize the difference in stability between the probe:target hybrid and probe:non-target hybrids.

Probes may be designed from the nucleic acids encoding a HIF-1a protein through methods known in the art. For instance, the G+C content of the probe and the probe length can affect probe binding to its target sequence. Methods to optimize probe specificity are commonly available in Sambrook et al. (2001) Molecular Cloning—A Laboratory Manual, Cold Spring Harbor Laboratory Press or Ausubel et al. (1995) Current Protocols in Molecular Biology, Greene Publishing.

Hybridization conditions are modified using known methods, such as those described by Sambrook et al. and Ausubel et al. as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The glass wafer can then be exposed to total cellular RNA or polyA RNA from a sample under conditions in which the affixed sequences will specifically hybridize. Such solid supports and hybridization methods are widely available, for example, those disclosed in WO 95/11755. By examining for the ability of a given probe to specifically hybridize to an RNA sample from an untreated cell population and from a cell population exposed to the agent, agents which up or down regulate the expression of a nucleic acid encoding the HIF-1a protein are identified.

Hybridization for qualitative and quantitative analysis of mRNA may also be carried out by using a RNase Protection Assay (i.e., RPA, see Ma et al. (1996) Methods 10, 273-238). Briefly, an expression vehicle comprising cDNA encoding the gene product and a phage specific DNA dependent RNA polymerase promoter (e.g., T7, T3 or SP6 RNA polymerase) is linearized at the 3′ end of the cDNA molecule, downstream from the phage promoter, wherein such a linearized molecule is subsequently used as a template for synthesis of a labeled antisense transcript of the cDNA by in vitro transcription. The labeled transcript is then hybridized to a mixture of isolated RNA (i.e., total or fractionated mRNA) by incubation at 45° C. overnight in a buffer comprising 80% formamide, 40 mM Pipes (pH 6.4), 0.4 M NaCl and 1 mM EDTA. The resulting hybrids are then digested in a buffer comprising 40 μg/ml ribonuclease A and 2 μg/ml ribonuclease H. After deactivation and extraction of extraneous proteins, the samples are loaded onto urea/polyacrylamide gels for analysis.

In another assay format, cells or cell lines are first identified which express HIF-1a gene products physiologically. Cell and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Further, such cells or cell lines would be transduced or transfected with an expression vehicle (e.g., a plasmid or viral vector) construct comprising an operable non-translated 3′-promoter containing end of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag or other detectable marker. Such a process is well known in the art (see Sambrook et al. (2001) Molecular Cloning—A Laboratory Manual, Cold Spring Harbor Laboratory Press).

Cells or cell lines transduced or transfected as outlined above are then contacted with agents (e.g., 2-oxoacids or derivatives thereof) under appropriate conditions. For example, the agent in a pharmaceutically acceptable excipient is contacted with cells in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells will be disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample will be compared with a control sample where only the excipient or control agents (cystine, cysteine or histidine) is contacted with the cells and an increase or decrease in the immunologically generated signal from the agent-contacted sample compared to the control will be used to distinguish the effectiveness of the agent

Methods to Identify Agents that Modulate HIF-1 Activity

The present invention provides methods for identifying agents that modulate at least one activity of the HIF-1a protein. Such methods or assays may utilize any means of monitoring or detecting the desired activity.

In one format, the specific activity of the HIF-1a protein, normalized to a standard unit, between a cell population that has been exposed to the agent to be tested compared to an unexposed control cell population may be assayed. Cell lines or populations are exposed to the agent to be tested under appropriate conditions and time. Cellular lysates may be prepared from the exposed cell line or population and a control, unexposed cell line or population. The cellular lysates are then analyzed with the probe.

Antibody probes can be prepared by immunizing suitable mammalian hosts utilizing appropriate immunization protocols using the proteins of the invention or antigen-containing fragments thereof. To enhance immunogenicity, these proteins or fragments can be conjugated to suitable carriers. Methods for preparing immunogenic conjugates with carriers such as BSA, KLH or other carrier proteins are well known in the art. In some circumstances, direct conjugation using, for example, carbodiimide reagents may be effective; in other instances linking reagents such as those supplied by Pierce Chemical Co. may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

While the polyclonal antisera produced in this way may be satisfactory for some applications, for pharmaceutical compositions, use of monoclonal preparations is preferred. Immortalized cell lines which secrete the desired monoclonal antibodies may be prepared using standard methods, see e.g., Kohler & Milstein (1992) Biotechnology 24, 524-526 or modifications which effect immortalization of lymphocytes or spleen cells, as is generally known. The immortalized cell lines secreting the desired antibodies can be screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies may be recovered from the culture supernatant or from the ascites supernatant Fragments of the monoclonal antibodies or the polyclonal antisera that contain the immunologically significant portion can be used as antagonists, as well as the intact antibodies. Use of immunologically reactive fragments, such as Fab or Fab′ fragments, is often preferable, especially in a therapeutic context, as these fragments are generally less immunogenic than the whole immunoglobulin.

The antibodies or fragments may also be produced, using current technology, by recombinant means. Antibody regions that bind specifically to the desired regions of the protein can also be produced in the context of chimeras with multiple species origin.

Antibody regions that bind specifically to the desired regions of the protein can also be produced in the context of chimeras with multiple species origin, for instance, humanized antibodies. The antibody can therefore be a humanized antibody or human a antibody, as described in U.S. Pat. No. 5,585,089 or Riechmann et al. (1988) Nature 332, 323-327.

Agents that are assayed in the above method can be randomly selected or rationally selected or designed. As used herein, an agent is said to be randomly selected when the agent is chosen randomly without considering the specific sequences involved in the association of the HIF-1a protein alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a non-random basis which takes into account the sequence of the target site or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site. Examples of rationally selected agents include, but are not limited to, cysteine, histidine and derivatives thereof.

The agents to be screened in the methods of the present invention can be, as examples, small molecules such as 2-oxoacids, peptides, peptide mimetics, antibodies, antibody fragments, small molecules, vitamin derivatives, as well as carbohydrates. Peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention are antibodies or fragments thereof that bind to HIF-1 protein hydroxylating enzymes or HIF-1b to inhibit their activity and hence, induce the activity of HIF-1a. Antibody agents can be obtained by immunization of suitable mammalian subjects with peptides, containing as antigenic regions as described herein, those portions of the protein intended to be targeted by the antibodies.

As used herein, “antibody” refers to a polypeptide comprising a framework region from an immunoglobulin gene or fragments thereof that specifically binds and recognizes an antigen. The immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50 to 70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies occur as intact immunoglobulins, as fragments produced by digestion with various peptidases, or as recombinant varieties, such as humanized antibodies or single chain antibodies. Thus, for example, pepsin digests an antibody below the disulfide linkages in the hinge region to produce F(ab)′₂ or a dimer of Fab which itself is a light chain joined to V_{H}-C_{H1} by a disulfide bond. The P(ab)′₂ may be reduced under mild conditions to break the disulfide linkage in the hinge region, thereby converting the F(ab)′₂ dimer into an Fab′ monomer. The Fab′ monomer is essentially Fab with part of the hinge region.

In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). Suitable leucine zipper sequences include the jun and fos leucine zippers and the GCN4 leucine zipper (Kostelney et al. (1992) J. Immunol. 148, 1547-1553; U.S. Pat. No. 6,133,426).

While various antibody fragments are defined in terms of the digestion of an intact antibody, one of skill will appreciate that such fragments may be synthesized de novo either chemically or by using recombinant methodology. Thus, the term antibody, as used herein, also includes antibody fragments either produced by the modification of whole antibodies, or those synthesized de novo using recombinant methodologies, such as recombinant IgG antibodies (U.S. Pat. Nos. 4,816,567 and 4,642,334; Queen et al. (1989) Proc. Natl Acad. Sci. USA 86, 10029-10033), single chain antibodies, or antibodies acquired by phage display, and monoclonal antibodies made by the hybridoma method (Kohler et al. (1975) Nature 256, 495).

The synthesis of single chain antibodies is described in U.S. Pat. No. 4,946,778, while single domain antibodies are described by Conrath et al. (2001) J. Biol. Chem. 276, 7346-7350 and Desmyter et al. (2001) J. Biol. Chem. 276, 26285-26290). Antibodies may also be produced by the phage display technique (Barbas et al. (2001) Phage Display: A Laboratory Manual, Cold Spring Harbor Laboratory Press; Kay et al. (1996) Phage Display of Peptides and Proteins: A Laboratory Manual, Academic Press). Techniques for the production of single chain antibodies (U.S. Pat. No. 4,946,778) can be adapted to produce antibodies to HIF-1b and HIF-1 hydroxylating enzymes. Also, transgenic mice, or other organisms such as other mammals, may be used to express humanized antibodies.

As used herein, a “chimeric antibody” is an antibody molecule in which part or all of the constant region is altered, with a replacement or exchange, so that the antigen binding site is linked to a constant region of a different class or antibody, or to an enzyme, hormone, protein toxin (U.S. Pat. No. 6,051,405), growth factor, or drug.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### EXAMPLES

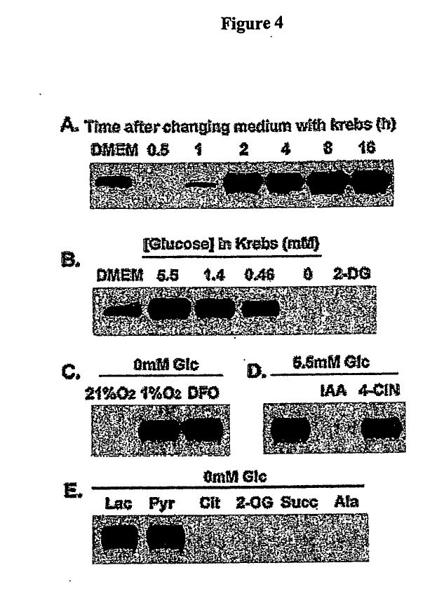

Recently, it was determined that human cells lines to display basally elevated levels of HIF-1a even under normoxic conditions (20% oxygen) (Lu et al. (2002) J. Biol. Chem. 277, 23 111-23115). The level of this basal HIF-1a expression varied with the specific cell line studied. Further exploration revealed that the differential basal expression of HIF-1 was a function of the different culture media that were being used to propagate the specific cell lines. Cells grown in media containing high glucose or added pyruvate appeared have detectable levels of HIF-1a under normoxia. In order to resolve the biochemical mechanisms underlying this phenomenon, we studied the human glioma cell line U-87 under conditions of carefully defined culture media. Thus we studied these cells while culturing them in freshly prepared Krebs buffer, all components of which were known to us. We found a time-dependent elevation of HIF-1a levels in these cells upon changing their media with fresh Krebs buffer (FIG. 4). Systematic removal of each component of the Krebs buffer revealed that the key component that led to accumulation of the HIF-1a protein was glucose. Thus, no increase in HIF-1 levels was seen in glucose-free Krebs, while glucose dose-dependently increased HIF-1a levels. Furthermore, the ability of glucose to stimulate HIF-1a could not be mimicked by the non-metabolizable glucose analog 2-deoxyglucose. Thus a metabolite of glucose was responsible for the accumulation of HIF-1a. Hypoxia and DFO, two known activators of HIF-1, could however still upregulate HIF-1a protein in the absence of glucose these results demonstrate that the glucose-mediated effects represented a novel mechanism distinct from those previously recognized.

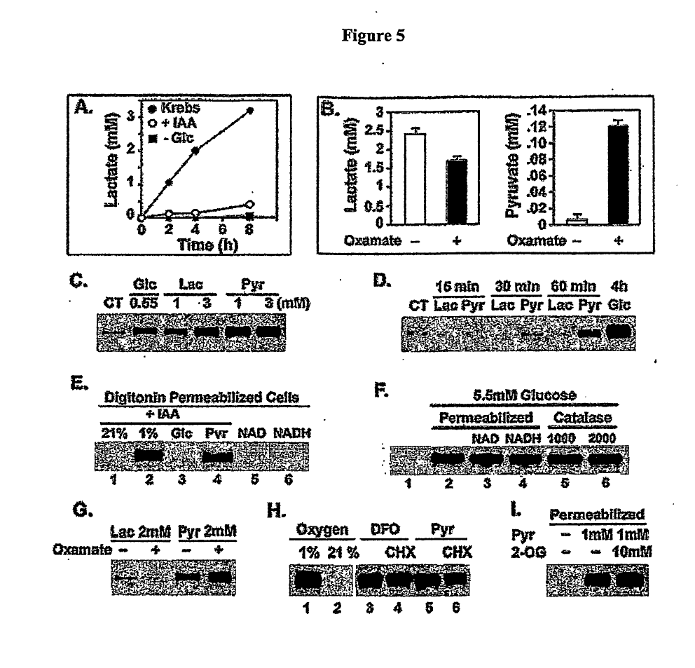

To precisely define the glucose metabolite mediating HIF-1a accumulation, we utilized pharmacological inhibitors of glycolysis as well as the direct addition of different glucose metabolites to cells (FIG. 4). Iodoacetamide, an inhibitor of glyceraldehyde phosphate dehydrogenase (GAPDH) completely blocked the ability of Krebs buffer to stimulate HIF-1a accumulation. Cinnamate, an inhibitor of pyruvate and lactate transport across mitochondrial and plasma membranes, did not prevent the effect of Krebs buffer on HIF-1a accumulation. These results narrowed down the responsible glucose metabolite to the steps after GAPDH. Addition of pyruvate and lactate in glucose-free Krebs was then found to activate HIF-1a directly while several pyruvate metabolites such as citrate, 2-oxoglutarate, succinate, and alanine were without effect. Lactate and pyruvate are highly produced by human cell line such as the U87 glioma cells that we primarily studied (FIG. 5). Lactate and pyruvate are also intercovertible via the enzyme lactate dehydrogenase (LDH). Glucose metabolism to pyruvate raises the cellular NADH/NAD ratio while pyruvate conversion to lactate lowers this ratio. To rule out the possibility that a change in NADH or NAD levels was responsible for elevation of the HIF-1a protein we utilized cells permeabilized with mild detergent (digitonin) treatment these preparations were able to induce HIF-1a by all known mediators. Direct addition of 3 mM NAD or NADH had no effect on HIF-1a levels, demonstrating that NADH/NAD ratios were not responsible for the HIF-1a activation by glucose metabolism. We utilized the LDH inhibitor oxamate to more specifically implicate pyruvate in HIF-1a activation. Oxamate blocked the ability of lactate to stimulate HIF-1a accumulation while potentiating the effect of pyruvate. These results demonstrated thet although lactate can stimulate HIF-1a accumulation, it must first be converted into pyruvate. Thus pyruvate was the major glucose metabolite responsible for stimulating HIF-1a accumulation. Pyruvate appears to enhance the accumulation of HIF-1a by inhibiting its degradation in a manner resembling the inactivation of the HPH enzymes. This was demonstrated in FIG. 5 by the observation that pyruvate maintained elevated HIF-1a levels in the absence of protein synthesis. Normally, HIF-1a has a very short half life and is degraded within minutes of being synthesized in the presence of oxygen and iron.

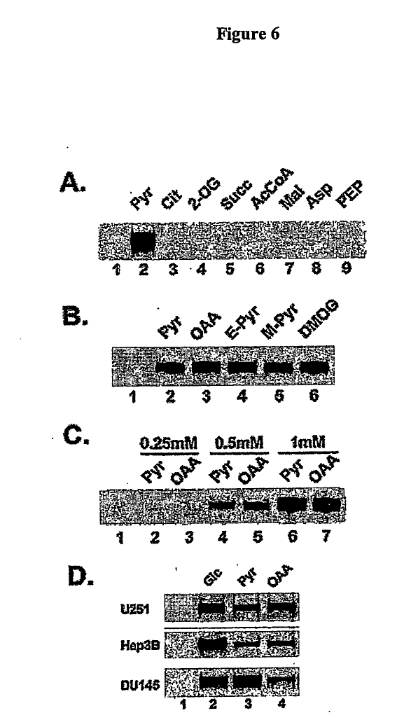

In evaluating other cellular metabolites we found that the structural requirements for HIF-1 activitating metabolites were quite specific. We found that oxaloacetate, a major metabolite of pyruvate and a Krebs cycle intermediate, is also a potent and effective inducer of HIF-1a protein (FIG. 6). Oxaloacetate and pyruvate can be interconverted via several metabolic routes. Despite this, both of these glucose metabolites appear sufficient to potently induce HIF-1a levels in many cell types.

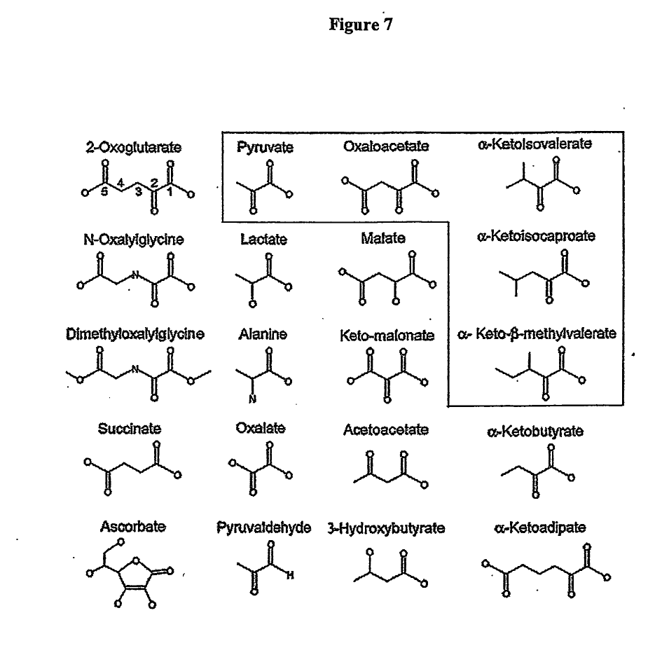

The structural diagrams in FIG. 7 are also revealing from the standpoint of future drug development based upon 2-oxoacids. We have shown that while oxaloacetate can activate HIF-1, succinate and 2-oxoglutarate cannot This points out the importance of the 2-oxo group in mediating HIF-1 activation, yet also shows the importance of appropriately positioned groups at the 4 and 5 positions. That pyruvate can activate HIF-1 shows that the minimal features we have determined to be required so far in activating HIF-1 are the 2-oxo group and a methyl group at the 3 position. Citrate, which has a carboxyl group at position 3 is ineffective as is malate. Simple biochemical derivative of pyruvate and acetate can be screened for the activation of HIF-1 in glucose-free media exactly as we have demonstrated above. This may allow for the development of simple drugs far more potent and stable than pyruvate for regulating hypoxic gene expression. Thus, our elucidation of the regulation of HIF-1 by small 2-oxoacids can be utilized to develop drugs that induce physiological responses, which improve survival under hypoxia. Note that the ethyl- and methylpyruvate derivatives that we have identified as HIF-1 activators are already being proposed for use in other clinical applications (Chang et al. (2003) Diabetologia. 46, 1220-1227, Fink (2003) Crit Care Med. 31(Suppl), S51-56)

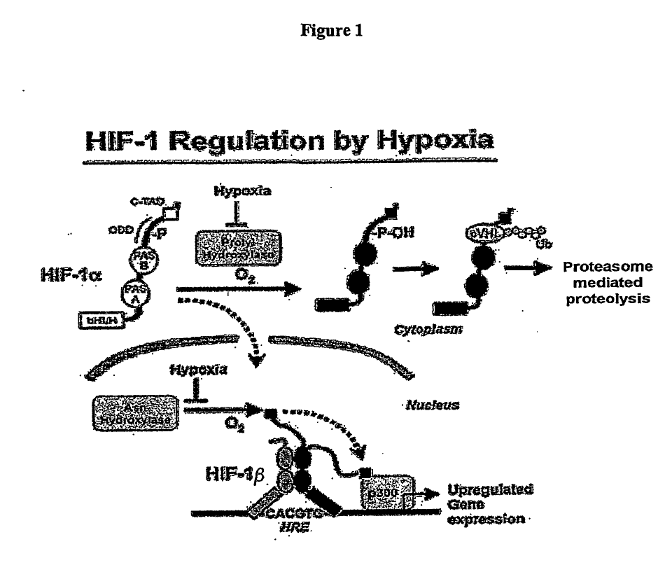

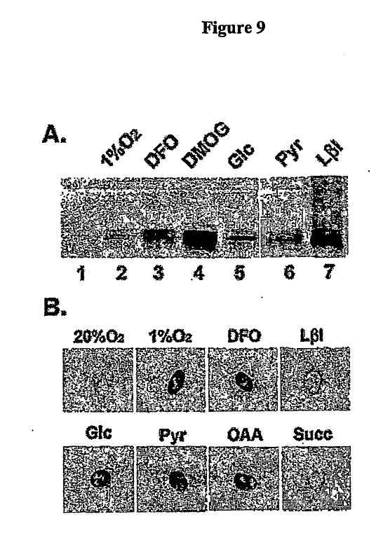

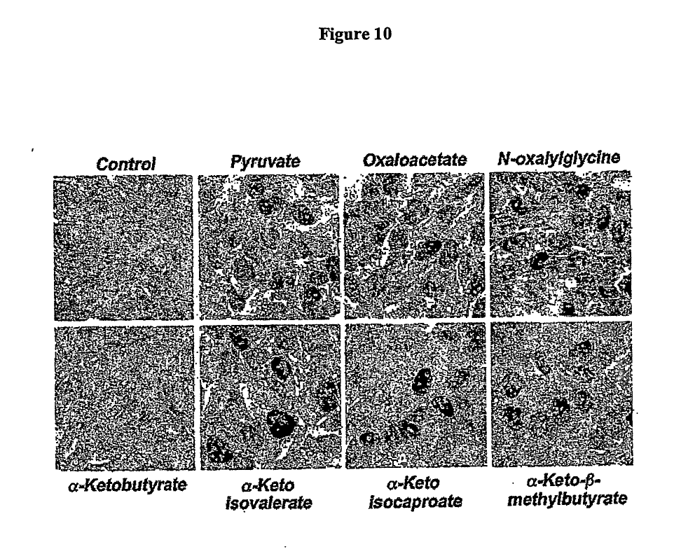

In order to investigate the mechanism of action of the 2-oxoacid HIF inducers that we identified we first determined their effects upon cellular ATP levels. As shown in FIG. 8A, during the four to eight hour culture periods used in our studies, addition of glucose or glucose metabolites to our glucose-free buffer does not have significant effects upon cellular ATP levels. Note that the effects of glucose, pyruvate, and oxaloacetate on ATP levels are not significantly different from those of 2-oxoglutarate and succinate despite their dramatically different effects on HIF-1a induction. Moreover, direct addition of ATP to permeabilized cells did not raise basal HIF-1a levels (FIG. 8B). These data point to a mechanism of HIF induction distinct from a change in cellular phosphorlylation potential. As shown in the scheme in FIG. 1, cellular levels of HIF-1a can be elevated by blockade of proteasomal activity, ubiquitinylation, or prolyl hydroxylation. Inhibition of the proteasome by lactacystin beta-lactone (Lbl) leads to accumulation of HIF-1a in its polyubiquitinylated form which generally appears as a smear of higher molecular weight species on western blots. This polyubiquitinylated form does not translocate to the nucleus and does not activate gene transcription. FIG. 9A shows that in whole cell extracts of U87 cells treated in Krebs buffer, HIF-1a induced by glucose or pyruvate has a molecular weight similar to that seen with induction by the HPH inhibitors hypoxia and desferrioxamine (DFO). The characteristic high molecular weight smear of poly-ubiquitinylated HIF-1a is only seen with lactacystin treatment. Cellular accumulation and nuclear translocation of HIF-1a can also be studied via immunohistochemistry. For this purpose we utilized U251 cells which are more adherent to cell culture dishes that the U87 cells. As shown in FIG. 9B, glucose, pyruvate, and oxaloacetate promote nuclear HIF-1a accumulation similar to hypoxia and DFO but are distinguished from lactacystin which only promotes cytosolic HIF-1a buildup. Succinate does not affect HIF-1a accumulation. Using this assay in U251 cells we also determined that endogenous branched chain 2-oxoacids can promote HIF-1a accumulation (FIG. 10).

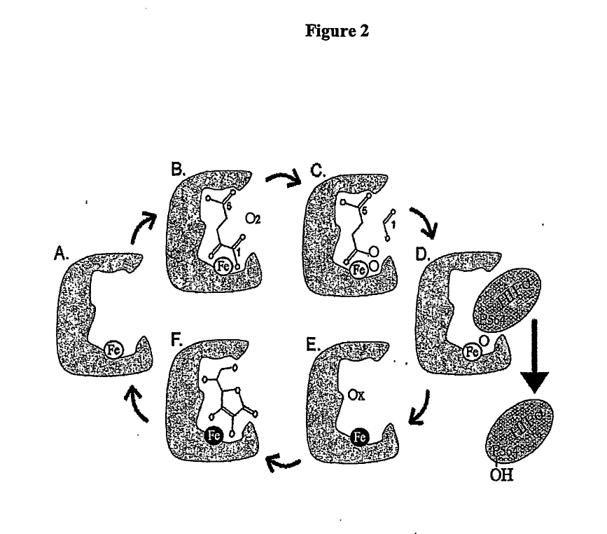

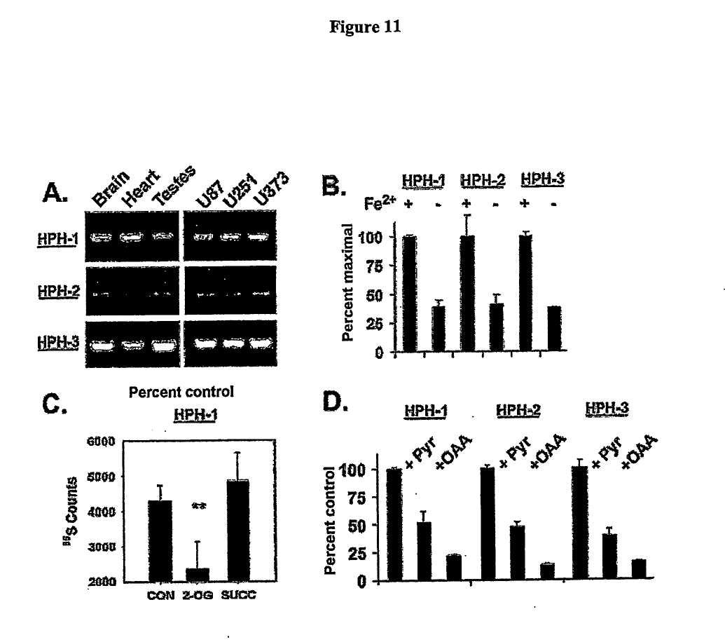

These data, together with the structural profile presented in FIG. 7 strongly suggested that the endogenous 2-oxoacid HIF-1a inducer may work by competing for the 2-OG binding site in HPHs in a manner similar to NOG or DMOG (see FIG. 2). We determined that all three known human HPH homologues were expressed within the cells we were studying (FIG. 11A). In order to investigate whether pyruvate or oxaloacetate could compete with the 2-OG binding site on HPH, we prepared an affinity column with 2-OG immobilized onto sepharose beads. We also prepared ³⁵S-labeled HPH homologues from expression plasmids using the rabbit reticulocyte culture system (Bruick et al. (2001) Science 294, 1337-1340). The 2-OG column allowed us to investigate the binding of ³⁵S-HPH as well as potential competitors of binding. This approach, which monitors step B in FIG. 2 has been used previously (Anzellotti et al. (2000) Arch. Biochem. Biophys. 382, 161-172). As shown in FIG. 11B all three ³⁵S-labeled HPH homologues bound to the immobilized 2-OG with more than half the binding showing a requirement for iron. Using ³⁵S-HPH-1 we also showed that its substrate 2-OG could displace this binding while the endproduct succinate could not (FIG. 11C). Using the iron dependent binding of ³⁵S-HPH we showed that both pyruvate and oxaloacetate could indeed compete for the 2-OG binding site (FIG. 11D).

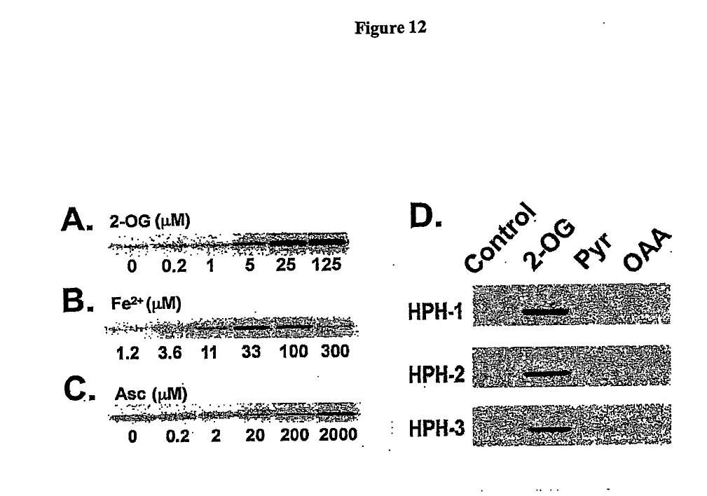

To directly evaluate the action of pyruvate and oxaloacetate on HPH activity (step D in FIG. 2) we utilized the commonly used ³⁵S-pVHL pulldown assay. This assay monitors the ability of HPH homologues to confer ³⁵S-pVHL binding activity onto a biotinylated 19mer peptide containing the key proline564 residue of the HIF-1a ODD (see FIG. 1). After incubating 1 μg amount of the peptide with in vitro translated HPH and the indicated reagents, ³⁵S-pVHL was added followed by the addition of Streptavidin-coated beads to pull down the HIF-1a peptide. The reaction was pelleted, and the pellet was washed and solubilized for SDS-PAGE analysis followed by autoradiography to reveal the captured ³⁵S-pVHL. Using each of the in vitro translated HPH homologues we first optimized assay conditions with respect to the required HPH substrates and co-factors. This optimization is shown for HPH-1 in FIG. 12A to C. Both 2-OG and iron were absolutely required for activity and although some activity was seen in its absence, ascorbate was also found to dose-dependently enhance activity. (D) Under conditions where all other reagents were kept constant (ascorbate=200 μM, ferrous sulfate=100 μM), 1 mM amounts of Pyr or OAA could not substitute for 100 μM 2-OG in catalyzing proline hydroxylation of HIF-1a peptide by-any of the three HPH homologues.

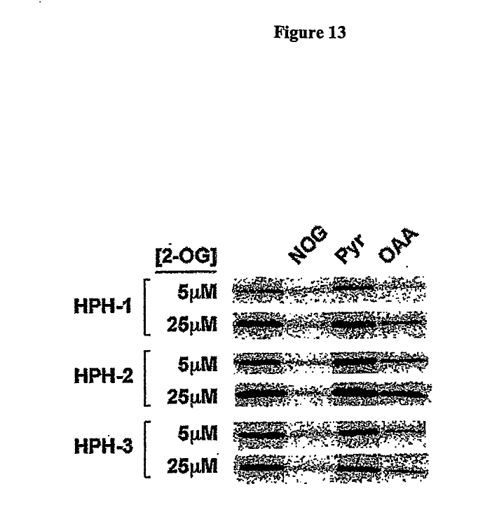

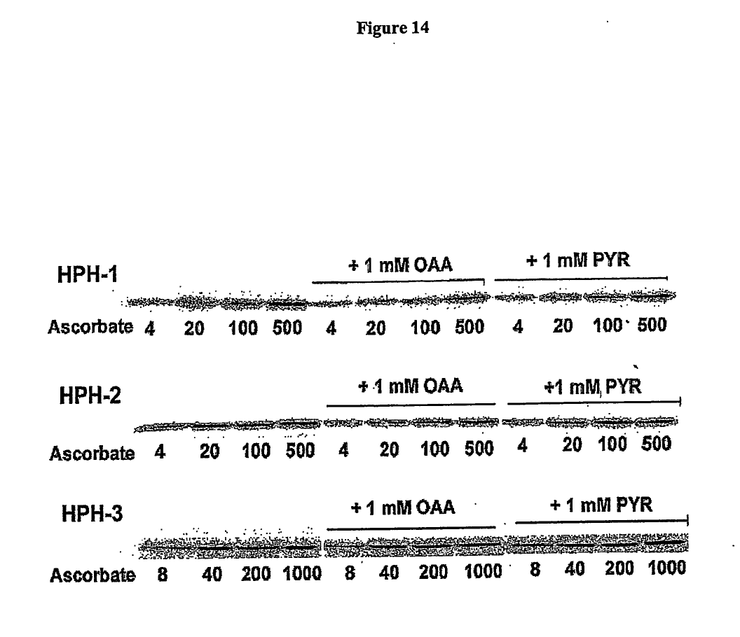

The ability of pyruvate and oxaloacetate to compete for the 2-OG binding site along with their inability to catalyze HIF-1a prolyl hydroxylation supported their potential role as 2-OG antagonists. Therefore, we examined their ability to inhibit 2-OG catalyzed HIF-1a hydroxylation using the ³⁵S-pVHL pull down assay. However, compared to the well-known HPH inhibitor N-oxalylglycine (NOG) we saw no inhibition by pyruvate little inhibition by oxaloacetate (FIG. 13). The assay conditions we utilized for this assay as per the literature are optimized to give ideal HPH activity. This includes the use of ascorbate at 2 mM levels so as to avoid the syn-catalytic inactivation described above. Cultured cells, on the other hand, do not routinely have extra ascorbate added to their media. We suspected that the discrepancy that we observed between the robust ability of pyruvate and oxaloacetate to induce HIF-1a accumulation with their poor ability to inhibit in vitro HPH activity resulted from inclusion of ascorbate in the in vitro assay. By varying the amount of ascorbate in the in vitro assay we indeed revealed an inhibitory effect of pyruvate on HPH-1 and HPH-2 activity that appeared to be ascorbate sensitive (FIG. 14).

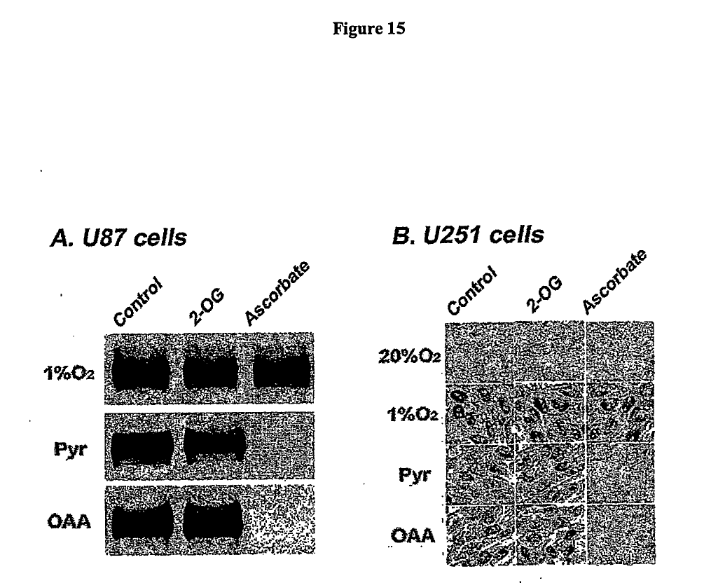

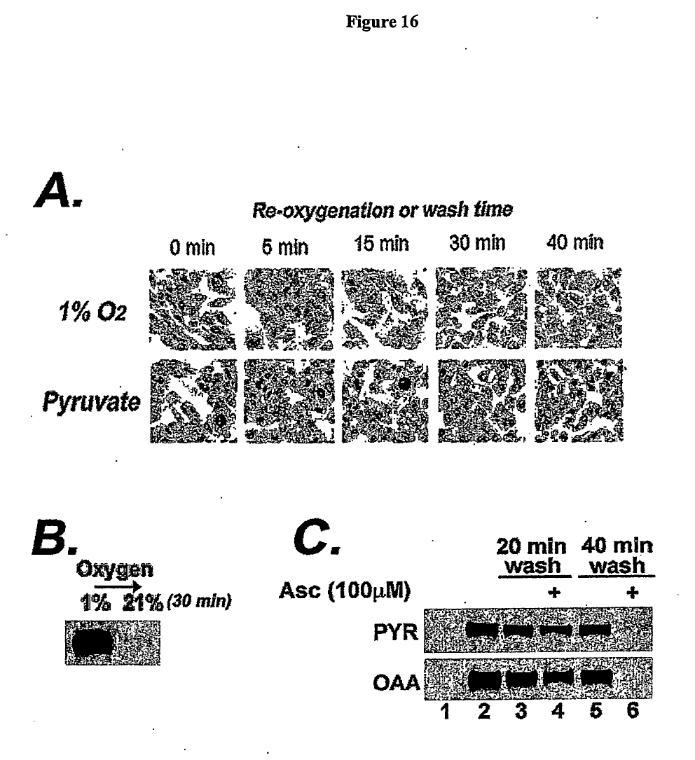

Interestingly, HPH-3 activity appeared to be insensitive to pyruvate or oxaloacetate. HPH-2 has been reported to constitute the major HPH activity of most cells. We therefore evaluated the ability of ascorbate to prevent HIF-1a accumulation by pyruvate and oxaloacetate in living cells. As shown in FIG. 15, inclusion of 100 μM ascorbate in the cultured cell experiments lead to complete inhibition of HIF-1a accumulation by pyruvate and ascorbate but not by hypoxia. Since ascorbate is known to reactivate the 2-oxoglutarate dependent dioxygenases following their syn-catalytic inactivation (FIG. 2E and F), our results suggested that pyruvate and oxaloacetate bind to HPHs and then inactivate them in an ascorbate reversible manner. The ascorbate sensitive inactivation is highly variable amongst family members of the 2-oxoglutarate dioxygenases and cannot be predicted without empirical data. To test this possibility we compared the reversibility of HIF-1a accumulation following induction with either hypoxia, pyruvate, or oxaloacetate. The rationale for these experiments stems from the well-known reversible inhibition of HPH activity by hypoxia (see FIG. 1). Thus HIF-1a accumulation and HIF activation during hypoxia is rapidly reversed upon re-oxygenation. FIG. 16A shows that the HIF-1a accumulation induced by hypoxia in U251 cells does indeed undergo a rapid decay upon re-oxygenation with no nuclear protein being detectable after 30 minutes of re-introducing oxygen. On the other hand, pyruvate induced HIF-1a accumulation persists well past 40 minutes after washing out the pyruvate. Similar results were seen in U87 cells via western blot analysis of nuclear extracts (FIG. 16B) with oxaloacetate. To investigate whether the 2-oxoacid induced persistence in HIF-1a accumulation was due to HPH inactivation we repeated this experiment with 100 μM ascorbate added to the wash buffer. As shown in FIG. 16C, ascorbate markedly enhanced the decay rate of HIF-1a

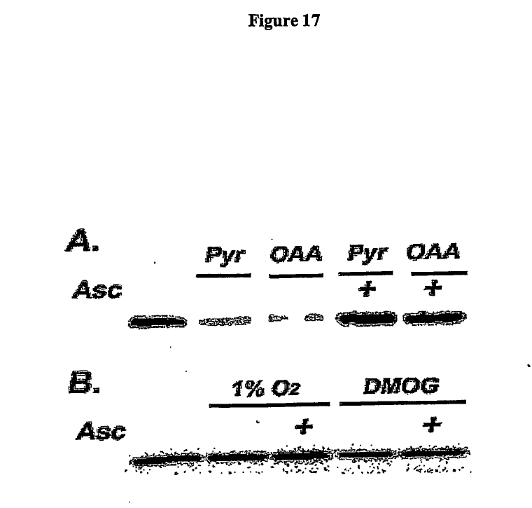

To directly determine whether HPH activity had been inactivated by pyruvate or oxaloacetate treatment, we evaluated the ability of U251 cell extracts from pyruvate or oxaloacetate treated cells to hydroxylate HIF-1a peptide using the ³⁵S-pVHL pulldown assay (Ivan et al. (2002) Proc. Natl. Acad. Sci. USA 99, 13459-13466). In these experiments, ascorbate was omitted from the in vitro portion of the assay. As shown in FIG. 17A, pyruvate and oxaloacetate pretreatment of cells clearly reduced HPH activity of cell extracts while the presence of ascorbate during the cell incubation period prevented this inhibition. No such pretreatment-induced inhibition was seen with hypoxia or DMOG (FIG. 17B).

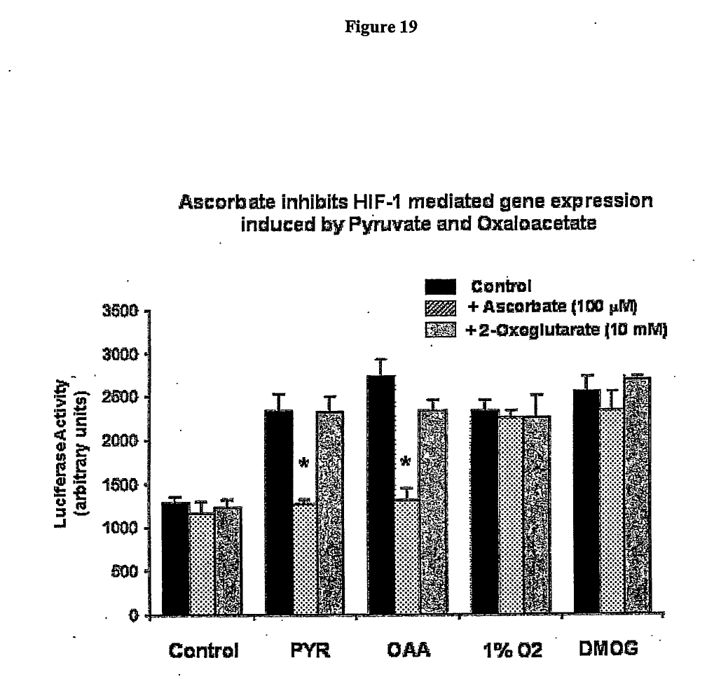

Effective gene expression by HIF not only involves HIF protein stabilization via inhibition of HPH enzymes but also HIF-1 binding to DNA, inhibition of FIH-1 activity, and gene transcription. The human gliomas that we utilized for most of our studies all express mRNA for FIH-1 (FIG. 18A). Furthermore, incubation of U87 cells under normoxia with pyruvate for four to six hour results in accumulation of HIF regulatory element DNA binding activity (FIG. 18B), and enhanced expression of several HIF regulated mRNA (FIG. 18C). In addition, pyruvate treatment of human Hep3B cells, which produce the well-known HIF regulated gene erythropoietin (Epo), resulted in a dose dependent increase in Epo levels (FIG. 18D). These data imply that pyruvate and perhaps other 2-oxoacids can also inhibit FIH-1 and fully activate the HIF signaling pathway shown in FIG. 1. To directly assess activation of gene containing an HRE regulated promoter by the glucose metabolites pyruvate and oxaloacetate, we used U373 glioma cells transfected with an HRE-green fluorescent (HRE-GFP) construct that responds to HIF activation. Hypoxia, DFO, and pyruvate all activated HRE-GFP expression as shown by the buildup of cellular fluorescence (FIG. 18E). In addition, U251 cells that were stably transfected with an HRE-luciferase construct also showed prominent activation of luciferase gene expression by hypoxia, pyruvate oxaloacetate and the ethyl- and methyl-pyruvate derivatives (FIG. 18F). Using the same HRE-luciferase expressing U251 cells we also were able to show that activation of HIF-dependent gene expression by pyruvate and oxaloacetate is distinguished from that by hypoxia or DMOG by its reversibility with ascorbate (FIG. 19).

Since most of the data presented here were obtained from experiments conducted with human cancer cell lines, we sought to determine whether pyruvate or oxaloacetate could activate the HIF pathway in normal cells and tissues. We therefore prepared primary cultures of rat cerebral cortical neurons and astrocytes and subjected these cells to a similar analysis as that for the cell lines described above. As shown in FIG. 20A rat neurons accumulate HIF-1a nuclear immunoreactivity upon four hours exposure to either 1% oxygen or to 3 mM pyruvate. Similarly, primary cultures of rat cerebrocortical astrocytes were also shown to induce HIF-1a upon treatment with hypoxia or with pyruvate (FIG. 20B). In order to determine the ability of pyruvate and oxaloacetate to activate HIF-1a in vivo we injected ten day old rats with intraperitoneal 500 mg/kg-doses of either pyruvate or oxaloacetate. We also subjected littermates to whole body hypoxia with either 8% oxygen or 0.1% carbon monoxide in air. Both of these paradigms have been shown to produce significant hypoxia and HIF-1 activation. Following four hours of each respective treatment, we harvested the animals' brains and prepared nuclear extracts for HIF-1 western blot analysis.

We also harvested kidneys for analysis of erythropoietin mRNA expression. As shown in FIG. 20C and D, rat brain displayed an increase in HIF-1a immunoreactivity following either hypoxia, pyruvate injection or oxaloacetate injection. FIG. 20E shows that renal erythropoietin gene expression was also stimulated by either hypoxia or oxaloacetate treatment. These results demonstrate the utility of using HPH (and presumably FIH-1) inactivating 2-oxoacids to regulate HIF-mediated gene expression.

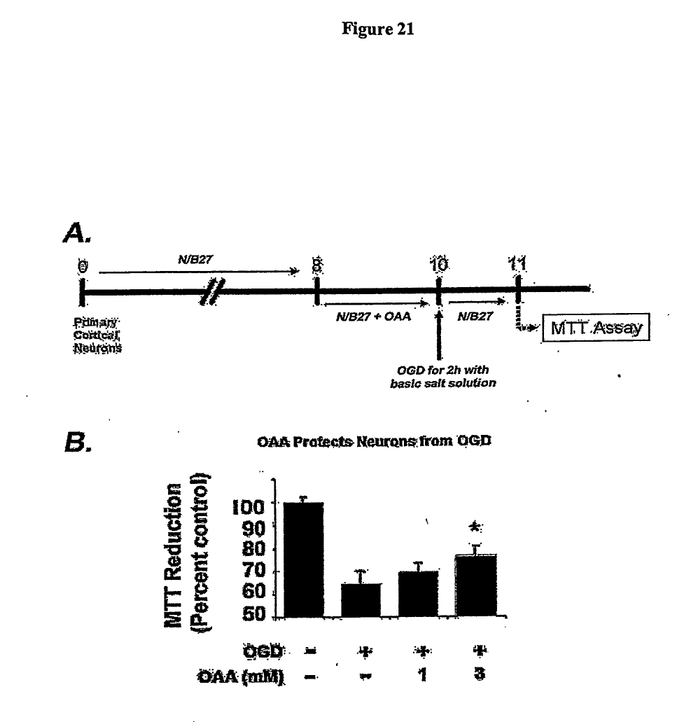

We have also completed work aimed at demonstrating beneficial physiological outcome from 2-oxoacid induced gene expression. One of these efforts involves the use of 2-oxoacids for hypoxic preconditioning. By inducing cytoprotective HIF-activated genes, 2-oxoacids such as pyruvate and oxaloacetate may be able to reduce the risk of stroke in elective cardiac or carotid surgery, lower ischemic bowel injury following gastrointestinal surgery, and also enhance the grafting efficiency of transplanted organs. To demonstrate the feasibility of such an approach, we have utilized a neuronal cell culture model of ischemic preconditioning. In this model, primary rat neuronal cultures are exposed to sublethal periods of oxygen and glucose deprivation (OGD), thereby mimicking ischemia. This brief OGD period is followed by return of cells to their regular culture conditions. A subsequent lethal period of OGD is then applied and the survival of OGD-preconditioned versus naïve cells is assessed via various cell survival assay to include the routinely used MTT reduction assay (Sawyer (1995) Clin. Exp. Pharmacol. Physiol. 22, 295-296). Recently, pharmaceutical efforts to induce ischemic or hypoxic preconditioning have been pursued in order to avoid the risk of exposing individuals to sublethal ischemia or hypoxia. One such recent effort has utilized the HIF-induced gene product erythropoietin, pretreatment with which shows remarkable OGD neuroprotection (Ruscher et al. (2002) J. Neurosci. 22, 10291-10301). We utilized the pretreatment paradigm used in this erythropoietin study to test whether oxaloacetate pretreatment could improve neuronal survival during OGD. The basic papradigm is shown in FIG. 21A. Rat cerebral cortex neurons are cultured for eight days in Neurobasal medium (N/B27). At that point oxaloacetate (OAA) or vehicle is added to the culture medium and the cells cultured for an additional 2 days. Following this period of treatment, neurons were made to undergo a two hour period of oxygen-glucose deprivation in which their media was replaced with a an isotonic salt solution lacking glucose. The neurons were also placed in an environment of 1% oxygen. This OGD paradigm results in significant delayed death of neurons one day later. As shown in FIG. 21B, pretreatment of neurons with 3 mM OAA improves survival in this paradigm. These results suggest that 2-oxoacids such as pyruvate and OAA that are capable of inducing HIF by inactivating HPHs can be used to induce hypoxic gene expression for therapeutic purposes.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

1. A method for activating HIF-1 mediated gene expression in a cell, comprising administering to said cell a composition comprising at least one 2-oxoacid selected from the group consisting of pyruvate, oxaloacetate, alpha-ketoisovalerate, alpha-ketoisocaproate, alpha-keto-beta-methylvalerate, methyl esters thereof, ethyl esters thereof and glycerol esters thereof.

2. The method as recited in claim 1, wherein said mammal is a human.

3. The method as recited in claim 1, wherein said HIF-1 mediated gene expression includes activation of expression of at least one gene selected from the group consisting of genes encoding vascular endothelial growth factor (VEGF), glucose transporter isoform 3 (Glut-3), aldolase A (aldo A) and erythropoietin.

4. The method as recited in claim 1, wherein said 2-oxoacid inhibits hydroxylation of HIF-1 in said cell.

5. The method as recited in claim 4, wherein said hydroxylation is mediated by a prolyl hydroxylase or an asparagine hydroxylase.

6. A method for inducing hypoxic adaptation in a mammal in need of such adaptation, comprising administering to said mammal a composition comprising at least one 2-oxoacid selected from the group consisting of pyruvate, oxaloacetate, alpha-ketoisovalerate, alpha-ketoisocaproate, alpha-keto-beta-methylvalerate, methyl esters thereof, ethyl esters thereof and glycerol esters thereof.

7. The method as recited in claim 6, wherein said mammal is a human.

8. The method of claim 6, wherein said human is at risk of heart attack, stroke or pregnancy-associated eclampsia.

9. The method of claim 7, wherein said human suffers from asthma, diabetes, epilepsy, anemia or cardiac arrythmias.

10. The method of claim 7, wherein said human has been exposed to high altitude or smoke inhalation.

11. A method of promoting tissue neovascularization in a mammal comprising administering to said patient a composition comprising at least one 2-oxoacid selected from the group consisting of pyruvate, oxaloacetate, alpha-ketoisovalerate, alpha-ketoisocaproate, alpha-keto-beta-methylvalerate, methyl esters thereof, ethyl esters thereof and glycerol esters thereof.

12. The method of claim 11, wherein said mammal is a human.

13. The method of claim 12, wherein said human has a peripheral vascular disease selected from the group consisting of atherosclerosis, vasculitis, phlebitis and thrombosis.

14. The method of claim 12, wherein said human is in need of wound or burn healing.

15. The method of claim 14, wherein said composition is applied topically.

16. A method for accelerating the development of proper oxygen homeostasis in a fetus comprising administering to a pregnant human a composition comprising at least one 2-oxoacid selected from the group consisting of pyruvate, oxaloacetate, alpha-ketoisovalerate, alpha-ketoisocaproate, alpha-keto-beta-methylvalerate, methyl esters thereof, ethyl esters thereof and glycerol esters thereof.

17. The method of claim 16 where said pregnant human is at risk for premature delivery.

18. A method for protecting a mammal against radiation comprising administering to said mammal a composition comprising at least one 2-oxoacids selected from the group consisting of pyruvate, oxaloacetate, alpha-ketoisovalerate, alpha-ketoisocaproate, alpha-keto-beta-methylvalerate, methyl esters thereof, ethyl esters thereof, and glycerol esters thereof.

19. The method as recited in claim 18, wherein said composition is administered before exposure to radiation, during exposure to radiation or after exposure to radiation.

20. The method as recited in claim 19, wherein said composition is administered one hour after exposure to radiation.

21. The method as recited in claim 19, wherein said composition is administered four hours after exposure to radiation.

22. The method as recited in claim 19, wherein said composition is administered twenty-four hours after exposure to radiation.

23. The method as recited in claim 18, wherein said mammal is a human.

24. The method as recited in claim 1 wherein said administering to said mammal of said composition is accomplished by at least one method selected from the group consisting of oral administration, mucosal administration, ocular administration, subcutaneous injection, transdermal administration, and combinations thereof.

25. The method as recited in claim 24, wherein said mucosal administration is selected from the group consisting of buccal, endotracheal, nasal, pharyngeal, rectal, sublingual, vaginal, and combinations thereof.

26. The method as recited in claim 24, wherein for said buccal, endotracheal, nasal, pharyngeal, sublingual, and combinations thereof administration, said composition is in a physical form selected from the group consisting of emulsion, gum, lozenge, spray, tablet and an inclusion complex.

27. The method of claim 26, wherein for said rectal and said vaginal administration, said composition is in a physical form selected from the group consisting of cream, douche, enema and suppository.

28. The method as recited in claim 24, wherein said composition for said nasal administration is selected from the group consisting of sniffing powder, and nasal spray.

29. The method as recited in claim 24, wherein said composition for said oral administration is selected from the group consisting of incorporation in food, incorporation into a dietary supplement, incorporation in a drink or powder to be mixed with water or other liquid, chewable tablet or capsule, swallowable tablet, capsule, caplet or softgel, Q-melt strip, bar, lozenge and gum.

30. The method as recited in claim 24, wherein said composition for said ocular administration is selected from suspension, solution and spray.

31. The method as recited in claim 24, wherein said composition for said subcutaneous administration is an incorporation in a pharmaceutically acceptable and injectable carrier.

32. The method as recited in claim 24, wherein said composition for transdermal administration is an incorporation into a lipophilic carrier with a physical form of a topical créme or a physical form of an adhesive patch.

33. The method as recited in claim 24, wherein said composition is administered repetitively with time intervals in the range of from about one hour to about forty-eight hours.
